(19) United States
(12) Patent Application Publication
(10) Pub. No.: US 2013/0200268 A1
(43) Pub. Date: Aug. 8, 2013

(54) ELECTRONICS FOR DETECTION OF A PROPERTY OF A SURFACE

(71) Applicant: MC10, Inc., Cambridge, MA (US)

(72) Inventors: Conor Rafferty, Newton, MA (US); Yung-Yu Hsu, Cambridge, MA (US); Benjamin Schlatka, Lexington, MA (US); Gilman Callsen, Charlottesville, VA (US)

(73) Assignee: MC10, Inc., Cambridge, MA (US)

(21) Appl. No.: 13/631,739

(22) Filed: Sep. 28, 2012

Related U.S. Application Data
(60) Provisional application No. 61/540,444, filed on Sep. 28, 2011.

(51) Int. Cl. A61B 5/00

(52) U.S. Cl. CPC A61B 5/441
USPC 250/372; 250/206; 257/461; 257/77; 257/76

(57) ABSTRACT

Apparatus are provided for monitoring a condition of a surface based on a measurement of a property of the surface using a sensor. In an example, the property is performed using an apparatus disposed above the tissue, where the apparatus includes at least one coil structure formed from a conductive material, at least one other component, and at least one cross-link structure physically coupling a portion of the at least one coil structure to a portion of the at least one other component, the at least one cross-link structure being formed from a flexible material. The at least one other component can be a sensor component or a processor unit.

### CROSS-REFERENCES TO RELATED APPLICATIONS

This application claims priority to U.S. provisional application Ser. No. 61/540,444, filed Sep. 28, 2011, entitled “METHODS, APPARATUS AND SYSTEMS FOR MONITORING UV AND SUNLIGHT EXPOSURE,” which is hereby incorporated herein by reference in its entirety.

### BACKGROUND

Effort is being made to develop electronics for application in monitoring properties of a surface, including in the field of skin care and skin health. For example, skin cancer is the most commonly diagnosed type of cancer and the majority of skin cancer can be linked to over-exposure to ultraviolet (UV) rays from the sun or sun-beds. Education may assist in the prevention of overexposure to UV electromagnetic rays, reducing the risk of skin cancer.

Tissue hydration is the process of absorbing and retaining water in biological tissues. In humans, a significant drop in tissue hydration can lead to dehydration and may trigger other serious medical conditions. Dehydration may result from loss of water itself, loss of electrolytes, and/or a loss of blood plasma. Previous techniques for monitoring tissue hydration have applied, e.g., an ultrasonic hydration monitor that employs ultrasound velocity to calculate hydration level. The ultrasound hydration monitor is generally attached to tissue such as muscles. The device generally uses a rigid frame to maintain a constant distance between an ultrasound transducer and a receiver.

The use of electronics in some medical-related applications can be hampered by the boxy, rigid way that much electronics are designed and packaged. Biological tissue is mainly soft, pliable and curved. By contrast, boxy, rigid electronics can be hard and angular, which could affect the measurement of tissue.

Such rigid electronics also may limit applications in non-medical-based systems.

### SUMMARY

In view of the foregoing, it is recognized and appreciated herein that both sufficient comfort and accuracy are desirable attributes of techniques for monitoring parameters of a surface related to skin care or skin health, including an exposure of the skin to electromagnetic radiation or a hydration state of the skin, via conformal electronics.

Accordingly, methods, apparatus and systems disclosed herein provide for quantifying and tracking exposure to electromagnetic radiation (including visible and UV rays) of a surface such as tissue using conformal electronics. These example methods, apparatus and systems may be used to inform consumers of their personal UV exposure and possibly reduce over-exposure to UV rays.

The conformal electronics described herein also have applications in non-medical-based systems, such as for quantifying and tracking an amount of exposure to electromagnetic radiation of a surface of paper, wood, leather, fabric (including artwork or other works on canvas), a plant or a tool.

Various examples described herein are directed generally to tissue condition monitoring methods, apparatus, and systems applicable to both consumer and military markets, which can provide real-time feedback as well as portability. The tissue condition can be state of hydration or disease state. In some examples, the methods, apparatus and systems are based at least in part on measuring properties of a surface (such as but not limited to the skin and underlying tissue), to provide an indication of the exposure of the surface to electromagnetic radiation, the SPF factor of a product, or a condition of the surface according to the principles described herein.

Accordingly, an apparatus for monitoring exposure of a surface to electromagnetic radiation is described. The apparatus includes a flexible substrate, at least one sensor component disposed on the flexible substrate, and at least one processing unit in communication with the at least one sensor component, and at least one cross-link structure physically coupled to a portion of the at least one processing unit and/or to a portion of the at least one sensor component, the at least one cross-link structure being formed from a dielectric material. The at least one sensor component measures an amount of electromagnetic radiation incident on the at least one sensor component, the electromagnetic radiation having frequencies in the visible or ultraviolet regions of the electromagnetic spectrum. The measure of the amount of electromagnetic radiation incident on the at least one sensor component provides an indication of an amount of exposure of the surface to the electromagnetic radiation.

In an example, the at least one cross-link structure physically couples a portion of the at least one processing unit to a portion of the at least one sensor component.

The apparatus can further include a memory in communication with the at least one sensor component, wherein the memory stores data indicative of measurements of the amount of electromagnetic radiation incident on the at least one sensor component.

The apparatus can further include a memory in communication with the at least one sensor component, where the memory stores machine readable instructions which, when executed, cause the at least one processing unit to analyze the measure of the amount of electromagnetic radiation incident on the at least one sensor component to provide the indication of the amount of exposure of the surface to the electromagnetic radiation.

The apparatus can further include at least one coil structure formed from a conductive material, and a radio-frequency component in communication with the at least one coil structure and/or the at least one processing unit, where the radio-frequency component transmits the measure of the amount of electromagnetic radiation incident on the at least one sensor component and/or the indication of an amount of exposure of the surface to the electromagnetic radiation using the at least one coil structure.

The radio-frequency component can be a BLUETOOTH® component.

The apparatus can further include at least one brace structure formed from a dielectric material, where the at least one cross-link structure physically couples a portion of the at least one processing unit and/or a portion of the at least one sensor component to the at least one brace structure.

The at least one brace structure and the at least one cross-link structure can be formed from the same material or formed from different materials. The at least one brace structure may surround the at least one processing unit and/or the at least one sensor component.

The flexible substrate and the at least one cross-link structure can be formed from the same material or formed from different materials. The flexible substrate and the at least one cross-link structure can be formed from a same polymer. The flexible substrate has a Young's modulus of less than about 10 GPa.

The apparatus can further include an encapsulation layer disposed over at least a portion of the at least one sensor component and/or at least a portion of the at least one processing unit.

The at least one sensor component and the at least one processing unit can be positioned at or near a midpoint of a depth of the apparatus.

The encapsulation layer can have a Young's modulus less than about 100 MPa.

Portions of the encapsulation layer can include an adhesive, where the adhesive attaches the portions of the encapsulation layer to the surface.

The encapsulation layer can be formed from a polymer.

The at least one sensor component can be a photodetector including a p-n junction.

The apparatus can further include at least one filter disposed above the at least one sensor component, where a measure of the electromagnetic radiation using the at least one filter and the at least one sensor component provides a measure of the amount of ultraviolet-A electromagnetic radiation and/or ultraviolet-B electromagnetic radiation incident on the surface.

The at least one sensor component can be at least partially embedded in the flexible substrate.

The at least one sensor component can include two sensor component, where one of the two sensor components can be stacked above the other of the two sensor components to provide a stacked sensor component.

A comparison of a measure of the electromagnetic radiation using the stacked sensor component to a measure of the electromagnetic radiation using another of the at least one sensor components provides a measure of the amount of ultraviolet-A electromagnetic radiation and/or ultraviolet-B electromagnetic radiation incident on the surface.

The at least one sensor component can include a photodetector.

The at least one sensor component can be at least one of a silicon-based photodetector, a silicon carbide-based photodetector, a germanium-based photodetector, a gallium nitride-based photodetector, an indium gallium nitride-based photodetector and an aluminum gallium nitride-based photodetector.

The surface can be a portion of a tissue, a fabric, a plant, an artwork, paper, wood, or a tool or piece of equipment.

The surface can be a portion of a tissue, where the measure of the amount of exposure of the surface of the tissue to the electromagnetic radiation provides a measure of a level of SPF protection of the tissue.

The at least one sensor component can include at least two sensor components, where an ultraviolet filter can be disposed above at least one of the at least two sensor components, where a comparison of a measure of the electromagnetic radiation using the sensor component including the ultraviolet filter to a measure of the electromagnetic radiation using another of the at least one sensor components having no ultraviolet filter provides the measure of a level of SPF protection of the tissue.

The apparatus can further include at least one amplifier in electrical communication with the at least one sensor component.

Also described herein is a system for monitoring exposure of a surface to electromagnetic radiation. The system includes at least one apparatus according to a principle described herein, and a reader device. The reader device receives from the at least one apparatus the data indicative of the measure of the amount of electromagnetic radiation incident on the at least one sensor component and/or the indication of an amount of exposure of the surface to the electromagnetic radiation.

The reader device can include a coupling member, where the reader device receives the data indicative of the measure of the amount of electromagnetic radiation incident on the at least one sensor component and/or the indication of an amount of exposure of the surface to the electromagnetic radiation when the coupling member can be electrically coupled to a portion of the at least one apparatus.

The surface can be a portion of a tissue, a fabric, a plant, an artwork, paper, wood, or a tool or piece of equipment.

The reader device can be a near-field communication (NFC)-enabled handheld device.

In another example according to the principles herein, an apparatus for monitoring exposure of a surface to electromagnetic radiation. The apparatus can include at least one sensor component, at least one coil structure formed from a conductive material, and at least one cross-link structure physically coupling a portion of the at least one coil structure to a portion of the at least one sensor component, the at least one cross-link structure being formed from a flexible material. The at least one sensor component measures an amount of electromagnetic radiation incident on the at least one sensor component, the electromagnetic radiation having frequencies in the visible or ultraviolet regions of the electromagnetic spectrum. The measure of the amount of electromagnetic radiation incident on the at least one sensor component provides an indication of an amount of exposure of the surface to the electromagnetic radiation.

The at least one sensor component can be surrounded by the at least one coil structure.

The at least one sensor component can be positioned outside the at least one coil structure.

The surface can be a portion of a tissue, a fabric, a plant, an artwork, paper, wood, or a tool or piece of equipment.

The at least one sensor component can be surrounded by the at least one coil structure.

The measure of the amount of exposure of the tissue to the electromagnetic radiation provides a measure of a level of SPF protection of the surface.

The apparatus can further include at least one processing unit in communication with the at least one sensor component.

The at least one processing unit can be configured to analyze the measure of the amount of electromagnetic radiation incident on the at least one sensor component to provide the indication of the amount of exposure of the surface to the electromagnetic radiation.

The apparatus can further include a radio-frequency component in communication with the at least one coil structure and the at least one processing unit, where the radio-frequency component transmits the measure of the amount of electromagnetic radiation incident on the at least one sensor component and/or the indication of an amount of exposure of the surface to the electromagnetic radiation using the at least one coil structure.

The at least one coil structure can include at least one corrugated portion.

The at least one corrugated portion can include a zig-zag structure, a serpentine structure, a grooved structure, or a rippled structure.

The at least one coil structure can be polygonal-shaped, circular-shaped, square-shaped or rectangular-shaped.

The apparatus can further include a flexible substrate, where the at least one sensor component and the at least one coil structure can be disposed on the flexible substrate.

The flexible substrate can be a polymer.

The at least one cross-link structure can be formed from a polymer.

The flexible substrate and the at least one cross-link structure can be formed from the same material or from different materials.

The flexible substrate and the at least one cross-link structure can be formed from a same polymer.

The flexible substrate has a Young's modulus of less than about 10 GPa.

The at least one sensor component can include a photodetector.

The at least one sensor component can be at least one of a silicon-based photodetector, a silicon carbide-based photodetector, a germanium-based photodetector, a gallium nitride-based photodetector, an indium gallium nitride-based photodetector and an aluminum gallium nitride-based photodetector.

The apparatus can further include a filter coupled to the at least one sensor component, where the filter can be disposed at a region of the at least one sensor component where the electromagnetic radiation can be incident.

A measure of a change in current of the photodetector provides the measure of the amount of electromagnetic radiation incident on the at least one sensor component.

The at least one sensor component measures the amount of ultraviolet (UV) electromagnetic radiation incident on the at least one sensor component.

The at least one sensor component measures the amount of UVA or UVB electromagnetic radiation incident on the at least one sensor component.

The apparatus can further include an encapsulation layer disposed over at least a portion of the at least one sensor component and the at least one coil structure.

The encapsulation layer can have a Young's modulus less than about 100 MPa.

The at least one sensor component can be positioned at or near a midpoint of a depth of the apparatus.

Portions of the encapsulation layer can include an adhesive, where the adhesive attaches the portions of the encapsulation layer to the surface.

The encapsulation layer can be formed from a polymer.

The polymer can be a polyimide, where the at least one sensor component measures the amount of visible electromagnetic radiation incident on the apparatus.

The encapsulation layer can be formed from an elastomer.

The encapsulation layer and the at least one cross-ink structures can be formed from the same material.

In another example according to the principles herein, a system for monitoring exposure of a surface to electromagnetic radiation is described. The system includes at least one apparatus and at least one other component. The at least one other component can be at least one of a battery, a transmitter, a transceiver, an amplifier, a processing unit, a charger regulator for a battery, a radio-frequency component, a memory, an analog sensing block, and a temperature sensor.

According to the principles herein, a method for monitoring exposure of a surface to electromagnetic radiation is described. The method includes receiving data indicative of the amount of electromagnetic radiation incident on the at least one sensor component, where the data can be obtained using at least one apparatus described herein, and analyzing the data using at least one processor unit. The analysis provides indication of an amount of exposure of the surface to the electromagnetic radiation.

In an example, the analyzing the data can include comparing the data to a calibration standard, where the comparing provides the indication of the amount of exposure of the surface to the electromagnetic radiation.

In another example, the calibration standard can include a correlation between values of the data and the indication of the amount of exposure of the surface to the electromagnetic radiation.

According to the principles herein, an electromagnetic radiation sensor is described that includes a substrate having a surface that can be exposed to electromagnetic radiation in the visible and ultraviolet regions of the electromagnetic spectrum, an electron collector region disposed in the substrate, a hole collector region disposed in the substrate, and a potential well region disposed in the substrate and surrounding at least a portion of the electron collector region and at least a portion of the hole collector region.

The electron collector region can include a highly donor doped semiconductor material. The hole collector region can include a highly acceptor doped semiconductor material.

Where the potential well region includes a donor doped semiconductor material, the substrate can be a p-type semiconductor material. Where the potential well region includes an acceptor doped semiconductor material and the substrate can be a n-type semiconductor material.

Where the potential well region includes a donor doped semiconductor material, the substrate can be a p-type semiconductor material, and the potential well region includes a lower concentration of a dopant than the electron collector region.

The substrate can include silicon, silicon carbide, germanium, gallium nitride, indium gallium nitride, or aluminum gallium nitride.

Where the substrate includes silicon, silicon carbide, or germanium, the hole collector region can be formed from a highly acceptor doped region of the substrate, and the hole collector region can include a boron dopant or a gallium dopant.

Where the substrate includes silicon, silicon carbide, or germanium, the electron collector region can be formed from a highly donor doped region of the substrate, and the electron collector region can include a phosphorus dopant or an arsenic dopant.

Where the substrate includes silicon, silicon carbide, or germanium, the potential well region can be formed from a donor doped region of the substrate, where the potential well region has a lower concentration of dopant than the electron collector region, and where the potential well region can include a phosphorus dopant or an arsenic dopant.

Where the substrate includes silicon, silicon carbide, or germanium, the potential well region can be formed from an acceptor doped region of the substrate, where the potential well region has a lower concentration of dopant than the hole collector region, and where the potential well region can include a boron dopant or a gallium dopant.

The electron collector region can be disposed proximate to the surface of the substrate or embedded in the substrate.

The hole collector region can be disposed proximate to the surface of the substrate or embedded in the substrate.

The substrate can have a thickness of less than 1 micron, about 1 micron, about 2 micron, about 3 microns, about 5 microns, about 10 microns, or greater than about 10 microns.

The potential well region can have a thickness greater than the thickness of the electron collector region or the hole collector region.

The electron collector region can have a thickness of less than 1 micron, about 1 micron, about 2 microns, about 3 microns, or greater than about 3 microns. The hole collector region can have a thickness of less than 1 micron, about 1 micron, about 2 microns, about 3 microns, or greater than about 3 microns. The potential well region can have a thickness of less than 1 micron, about 1 micron, about 2 microns, about 3 microns, about 4 microns, or greater than about 4 microns.

A portion of the potential well can be disposed between the electron collector region and the hole collector region.

According to principles herein, a system is described that includes at least one coil structure formed from a conductive material, at least one other component surrounded by the at least one coil structure, and at least one cross-link structure physically coupling a portion of the at least one coil structure to a portion of the at least one other component, the at least one cross-link structure being formed from a flexible material. The at least one other component can be at least one of a battery, a transmitter, a transceiver, an amplifier, a processing unit, a charger regulator for a battery, a radio-frequency component, a memory, an analog sensing block, and a temperature sensor.

The system can further include at least one sensor component.

The at least one sensor component can be used to measure an amount of electromagnetic radiation incident on the at least one sensor component, the electromagnetic radiation having frequencies in the visible or ultraviolet regions of the electromagnetic spectrum.

The system can be disposed on a surface, where the measure of the amount of electromagnetic radiation incident on the at least one sensor component provides an indication of an amount of exposure of the surface to the electromagnetic radiation.

The at least one sensor component can be positioned external to the at least one coil structure, where the at least one sensor component can be electrically coupled to the at least one coil structure or to the at least one other component.

At least one other component or the at least one sensor component can be surrounded by the at least one coil structure.

The system can be disposed on a tissue, where the at least one sensor component measures a hydration level of the tissue.

The at least one other component can be a radio-frequency component and a processing unit, where the radio-frequency component can be in communication with the at least one coil structure and the at least one processing unit, where the radio-frequency component transmits data indicative of a measurement performed by the at least one sensor component.

The at least one sensor component can include a photodetector.

The at least one sensor component can be at least one of a silicon-based photodetector, a silicon carbide-based photodetector, a germanium-based photodetector, a gallium nitride-based photodetector, an indium gallium nitride-based photodetector and an aluminum gallium nitride-based photodetector.

The system can further include a filter coupled to the at least one sensor component, where the filter can be disposed at a region of the at least one sensor component where the electromagnetic radiation can be incident.

A measure of a change in current of the photodetector can be used to provide the measure of the amount of electromagnetic radiation incident on the at least one sensor component.

The system can be disposed on a surface, where the surface can be a portion of a tissue, a fabric, a plant, an artwork, paper, wood, or a tool or piece of equipment.

The at least one coil structure can include at least one corrugated portion. In an example, the at least one corrugated portion can include a zig-zag structure, a serpentine structure, a grooved structure, or a rippled structure.

The at least one coil structure can be polygonal-shaped, circular-shaped, square-shaped or rectangular-shaped.

The system can further include a flexible substrate, where the at least one sensor component and the at least one coil structure can be disposed on the flexible substrate.

The flexible substrate can be a polymer.

The at least one cross-link structure can be formed from a polymer.

The flexible substrate and the at least one cross-link structure can be formed from the same material or different materials.

The flexible substrate and the at least one cross-link structure can be formed from a same polymer.

The system can further include an encapsulation layer disposed over at least a portion of the at least one coil structure and the at least one other component.

The at least one sensor component can be positioned at or near a midpoint of a depth of the system.

The system can be disposed on a surface, where portions of the encapsulation layer include an adhesive, where the adhesive attaches the portions of the encapsulation layer to the surface.

The encapsulation layer can be formed from a polymer.

In an example, the analyzing the data can include applying an effective circuit model to the data, and where a value of a parameter of the model provides the indication of the condition of the tissue. In another example, the analyzing the data can include comparing the data to a calibration standard, and where the comparing provides the indication of the condition of the tissue.

The calibration standard can include a correlation between values of electrical measurement and the indication of the condition of the tissue.

The following publications, patents, and patent applications are hereby incorporated herein by reference in their entirety:

Kim et al., “Stretchable and Foldable Silicon Integrated Circuits,” Science Express, Mar. 27, 2008, 10.1126/science.1154367;

Ko et al., “A Hemispherical Electronic Eye Camera Based on Compressible Silicon Optoelectronics,” Nature, Aug. 7, 2008, vol. 454, pp. 748-753;

Kim et al., “Complementary Metal Oxide Silicon Integrated Circuits Incorporating Monolithically Integrated Stretchable Wavy Interconnects,” Applied Physics Letters, Jul. 31, 2008, vol. 93, 044102;

Kim et al., “Materials and Noncoplanar Mesh Designs for Integrated Circuits with Linear Elastic Responses to Extreme Mechanical Deformations,” PNAS, Dec. 2, 2008, vol. 105, no. 48, pp. 18675-18680;

Meitl et al., “Transfer Printing by Kinetic Control of Adhesion to an Elastomeric Stamp,” Nature Materials, January, 2006, vol. 5, pp. 33-38;

U.S. Patent Application publication no. 2010 0002402-A1, published Jan. 7, 2010, filed Mar. 5, 2009, and entitled “STRETCHABLE AND FOLDABLE ELECTRONIC DEVICES;”

U.S. Patent Application publication no. 2010 0087782-A1, published Apr. 8, 2010, filed Oct. 7, 2009, and entitled “CATHETER BALLOON HAVING STRETCHABLE INTEGRATED CIRCUITRY AND SENSOR ARRAY;”

U.S. Patent Application publication no. 2010 0116526-A1, published May 13, 2010, filed Nov. 12, 2009, and entitled “EXTREMELY STRETCHABLE ELECTRONICS;”

U.S. Patent Application publication no. 2010 0178722-A1, published Jul. 15, 2010, filed Jan. 12, 2010, and entitled “METHODS AND APPLICATIONS OF NON-PLANAR IMAGING ARRAYS;”

U.S. Patent Application publication no. 2010 027119-A1, published Oct. 28, 2010, filed Nov. 24, 2009, and entitled “SYSTEMS, DEVICES, AND METHODS UTILIZING STRETCHABLE ELECTRONICS TO MEASURE TIRE OR ROAD SURFACE CONDITIONS;”

PCT Patent Application publication no. WO2011/084709, published Jul. 14, 2011, entitled “Methods and Apparatus for Conformal Sensing of Force and/or Change in Motion;” and

U.S. Patent Application publication no. 2011 0034912-A1, published Feb. 10, 2011, filed Mar. 12, 2010, and entitled “SYSTEMS, METHODS, AND DEVICES HAVING STRETCHABLE INTEGRATED CIRCUITRY FOR SENSING AND DELIVERING THERAPY.”

U.S. Patent Application publication no US 2010-0298895-A1, published Nov. 25, 2010, and entitled “SYSTEMS, METHODS, AND DEVICES USING STRETCHABLE OR FLEXIBLE ELECTRONICS FOR MEDICAL APPLICATIONS.”

U.S. Patent Application publication no 2012-0065937-A1, published Mar. 15, 2012, and entitled “METHODS AND APPARATUS FOR MEASURING TECHNICAL PARAMETERS OF EQUIPMENT, TOOLS AND COMPONENTS VIA CONFORMAL ELECTRONICS.”

U.S. Patent Application publication no US 2012-0226130-A1, published Sep. 6, 2012, and entitled “SYSTEMS, METHODS, AND DEVICES HAVING STRETCHABLE INTEGRATED CIRCUITRY FOR SENSING AND DELIVERING THERAPY.”

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

The foregoing and other aspects, examples, and features of the present teachings can be more fully understood from the following description in conjunction with the accompanying drawings.

### BRIEF DESCRIPTION OF THE DRAWINGS

The skilled artisan will understand that the figures, described herein, are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIG. 1 shows a block diagram of a non-limiting example system, according to the principles herein.

FIG. 2 shows a block diagram of a non-limiting example system, according to the principles herein.

FIG. 3 shows a block diagram of a non-limiting example system, according to the principles herein.

FIG. 4 shows a block diagram of a non-limiting example system, according to the principles herein.

FIG. 5 shows a cross-section of an example apparatus or system, according to the principles described herein.

FIG. 6 shows a cross-section of an example layer structure, according to the principles herein.

FIGS. 7A-7D shows example apparatus or systems, according to the principles herein.

FIG. 8 shows non-limiting examples of tissue conditions that may be monitored using one or more of the apparatus described herein, according to the principles herein.

FIGS. 9A and 9B show the ultraviolet-A and ultraviolet-B wavelength regions, respectively, of response of example UV sensors, according to the principles herein.

FIG. 10 shows a table of non-limiting example values of parameters from an operation of an apparatus or system, according to the principles herein.

FIG. 11A shows non-limiting example apparatus, according to the principles described herein.

FIG. 11B shows non-limiting example apparatus, according to the principles described herein.

FIG. 12A shows non-limiting example apparatus, according to the principles described herein.

FIG. 12B shows non-limiting example apparatus, according to the principles described herein.

FIG. 13 shows an example apparatus, according to the principles herein.

FIG. 14 shows example measurement of the inductance (in units of pH) for a rectangular-shaped coils, according to the principles herein.

FIGS. 15A and 15B show an example implementation of a method for calibrating a measurement of a sensor component, according to the principles herein.

FIGS. 16A and 16B show an example implementation of a method for measurement of different UV blockers, according to the principles herein.

FIG. 17 shows an example photodetector, according to the principles herein.

FIG. 18 shows a non-limiting example photodetector, according to the principles herein.

FIG. 19 shows the absorption depth of electromagnetic radiation in a silicon substrate, according to the principles herein.

FIG. 20 shows the result of example measurements of a photodetector based on a silicon substrate, according to the principles herein.

FIG. 21 shows a non-limiting example of a hydration sensor, according to the principles herein.

FIG. 22 shows the hydration sensor of FIG. 21 electrically coupled to an apparatus, according to the principles herein.

FIG. 23 shows an example implementation of a structure as described in connection with FIG. 22, according to the principles herein.

FIGS. 24A-24I show a non-limiting example process for fabricating an apparatus or system, according to the principles herein.

FIG. 25 illustrates use of a patch with a handheld device for monitoring tissue condition, according to the principles herein.

### DETAILED DESCRIPTION

Following below are more detailed descriptions of various concepts related to, and examples of, methods and apparatus for measuring electrical properties of tissue. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

As used herein, the term “includes” means includes but not limited to, the term “including” means including but not limited to. The term “based on” means based at least in part on.

The apparatus and systems described herein provide technology platforms that use ultra-thin components linked with stretchable interconnects and embedded in low modulus polymers which provide a match to biological tissue and other types of surfaces. The technology platform implements high-performance active components in new mechanical form factors.

In an example, an apparatus and system described herein relates to the field of skin care using skin-mountable (epidermal) electronics. The apparatus, systems and methods described herein include sensors that can also be used to provide information in non-biological systems. In particular, the apparatus, systems and methods according to the principles described herein can be used to provide an indication of the exposure of a surface to electromagnetic radiation, the SPF factor of a product applied to the surface, or a condition of the surface. The surface can be of paper, wood, leather, fabric (including artwork or other works on canvas), a plant or a tool.

In non-limiting example, the technology platforms according to the principles described herein can be fabricated based on foundry complimentary metal-oxide-semiconductor (CMOS) wafers and transferred to polymer-based and/or polymer-coated carriers.

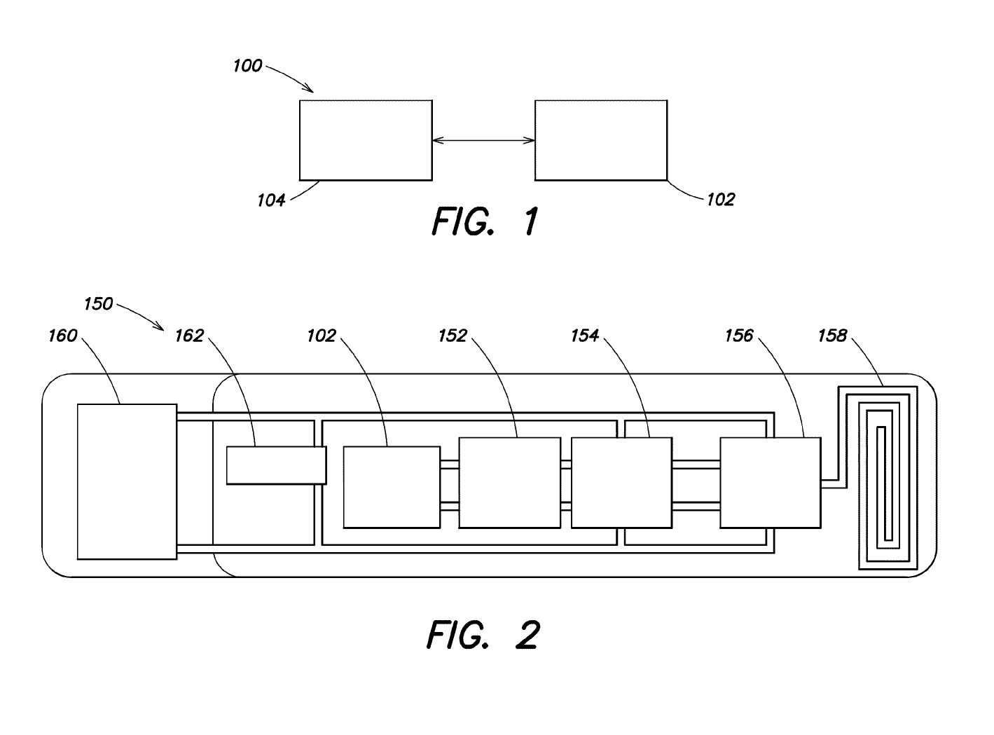

FIG. 1 shows a block diagram of a non-limiting example system according to the principles herein. The example system 100 includes at least one apparatus 102 that can be used to provide a measurement of a property of a surface. For example, the property can be an amount of electromagnetic radiation that the surface is exposed to. In this example, the at least one apparatus 102 can be configured as describe herein to perform a photo-detection measurement. As another example, the property can be a measure of an electrical property of the surface. In this example, the at least one apparatus 102 can be configured as describe herein to perform a capacitive-based measurement of the electrical properties of tissue (e.g., to provide a measure e of the state of hydration). The system 100 includes at least one other component 104 that is coupled to the at least one apparatus 102. In an example implementation, the at least one other component 104 can be a processing unit. In an example implementation, the at least one component 104 can be configured to supply power to the apparatus 102. For example, the at least one component 104 can include a battery or any other energy storage device that can be used to supply a potential. In an example implementation, the system 100 can include at least one component 104 for providing an indication of the measurement made by the apparatus. In an example implementation, the at least one component 104 can include at least one processor unit configured for analyzing the signal from the apparatus. In an example implementation, the at least one component 104 can be configured to transmit a signal from the apparatus to an external system or device. For example, the at least one component 104 can include a transmitter or a transceiver configured to transmit a signal including data measured by the apparatus measurement from the apparatus to a hand-held device or other computing device. Non-limiting examples of a handheld device include a smartphone, a tablet, a slate, an e-reader, a digital assistant, or any other equivalent device. As a non-limiting example, the hand-held device or other computing device can include a processor unit that is configured for analyzing the signal from the apparatus. The at least one other component 104 can be a temperature sensor.

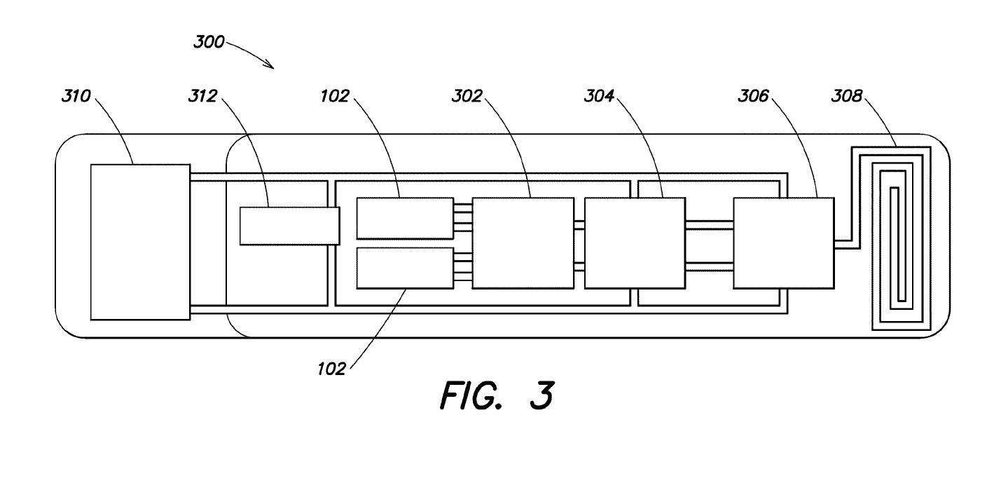

FIG. 2 shows a block diagram of a non-limiting example system 150 according another implementation of the principles herein. The example system 150 includes at least one apparatus 102 that can be used to perform a measurement of an amount of exposure of a surface to electromagnetic radiation or of the electrical properties of the surface through a capacitive-based measurement\. In the non-limiting example of FIG. 2, the at least one other component 104 includes an analog sensing block 152 that is coupled to the at least one apparatus 102 and at least one processor unit 154 that is coupled to the analog sensing block 152. The at least one other component 104 includes a memory 156. For example, the memory 156 can be a non-volatile memory. As a non-limiting example, the memory 156 can be mounted as a portion of a RF chip. The at least one other component 104 also includes a transmitter or transceiver 158. The transmitter or transceiver 158 can be used to transmit data from the apparatus 102 to a handheld device or other computing device (e.g., for further analysis). The example system 150 of FIG. 2 also includes a battery 160 and a charge regulator 162 coupled to battery 160. The charge regulator 162 and battery 160 are coupled to the processor unit 154 and memory 156.

A non-limiting example use of system 150 is as follows. Battery 160 provides power for the apparatus 102 to perform the measurements. The processor unit 154 activates periodically, stimulates the analog sensing block 152, which conditions the signal and delivers it to an A/D port on the processor unit 154. The data from apparatus 102 is stored in memory 156. In an example, when a near-field communication (NFC)-enabled handheld device is brought into proximity with the system 150, data is transferred to the handheld device, where it is interpreted by application software of the handheld device. The data logging and data transfer can be asynchronous. For example, data logging can occur each minute while data transfer may occur episodically.

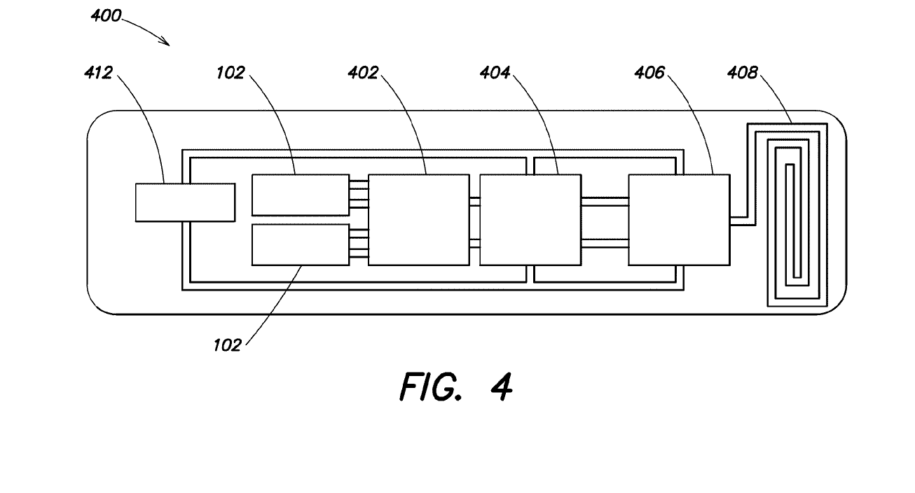

FIG. 3 shows a block diagram of a non-limiting example system 300 according another implementation of the principles herein. Example system 300 is configured for data logging. The example system 300 includes at least one apparatus 102 that can be used to perform a measurement of an amount of exposure of a surface to electromagnetic radiation. In the example depicted in FIG. 3, apparatus 102 includes a sensor component for detecting ultraviolet-A (UVA) electromagnetic radiation and another sensor component for measuring ultraviolet-B (UVB) electromagnetic radiation. In another example according to this implementation, apparatus 102 a capacitive-based measurement. In the non-limiting example of FIG. 3, the at least one other component 104 includes an analog sensing block 302 that is coupled to the at least one apparatus 102 and at least one processor unit 304 that is coupled to the analog sensing block 302. The at least one other component 104 includes a memory 306. For example, the memory 306 can be a non-volatile memory. As a non-limiting example, the memory 306 can be mounted as a portion of a RF chip. The at least one other component 104 also includes a transmitter or transceiver 308. The transmitter or transceiver 308 can be used to transmit data from the apparatus 102 to a handheld device or other computing device (e.g., for further analysis). The example system 300 of FIG. 3 also includes a battery 310 and a charge regulator 312 coupled to battery 310. The charge regulator 312 and battery 310 are coupled to the processor unit 314 and memory 316.

A non-limiting example use of system 300 is as follows. Battery 310 provides power for the apparatus 102 to perform the measurements. The processor unit 304 activates periodically, stimulates the analog sensing block 302, which conditions the signal and delivers it to an A/D port on the processor unit 304. The data from apparatus 102 is stored in memory 306. In an example, when a near-field communication (NFC)-enabled handheld device is brought into proximity with the system 300, data is transferred to the handheld device, where it is interpreted by application software of the handheld device. The data logging and data transfer can be asynchronous. For example, data logging can occur each minute while data transfer may occur episodically.

In an example method of use of a system that includes an apparatus described herein, a sensor component of the system can be maintained in a low power mode or a low operation mode. For example, the sensor component can be maintained in a “sleep” mode. At a specified interval of time, a processor unit of the system can include machine readable instructions that, when executed, causes the processor unit to periodically control one or more other components of the system and perform a measurement. For example, at regular periodic intervals of time, a microcontroller of the system can activate to cause the sensor to perform a sensor measurement (including an analog measurement). In a non-limiting example, the system includes a data logging component, and the processor unit causes the data from the measurement to be logged into a memory. In a non-limiting example, the system includes a radio-frequency component, and the processor unit causes the data from the measurement to be transferred to the radio-frequency component. In a non-limiting example, the radio-frequency component can include a Bluetooth component. In a non-limiting example, the system includes a coil structure, and the RF component transmits the data using the coil structure. In a non-limiting example, the data can be accesses or otherwise read-out using a near-field communication (NFC)-enabled handheld device that is brought in proximity with the system. In this example, the data can be read-out on-demand using the NFC-enabled handheld device.

As described in connection with FIG. 3, an example system according to the principles herein can be configured as a self-contained system with power and wireless communication for monitoring the property of a surface, such as but not limited to monitoring the amount of electromagnetic radiation it is exposed to, or the sweat level of the tissue (which can be related to its hydration level) and/or the disease of the tissue).

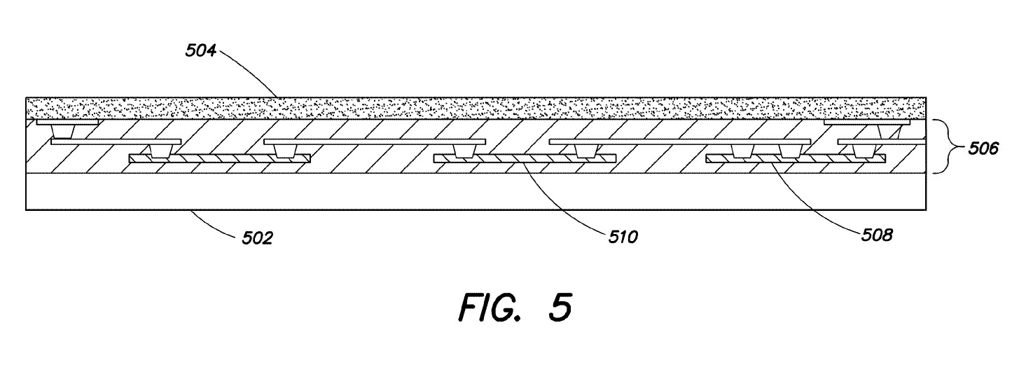

FIG. 4 shows a block diagram of a non-limiting example system 400 according another implementation of the principles herein. Example system 400 is configured without a power source. The example system 400 includes at least one apparatus 102 that can be used to perform a measurement of an amount of exposure of a surface to electromagnetic radiation. In the example depicted in FIG. 4, apparatus 102 includes a sensor component for detecting ultraviolet-A electromagnetic radiation and another sensor component for measuring ultraviolet-B electromagnetic radiation. In another example according to this implementation, apparatus 102 a capacitive-based measurement. In the non-limiting example of FIG. 4, the at least one other component 104 includes an analog sensing block 402 that is coupled to the at least one apparatus 102 and at least one processor unit 404 that is coupled to the analog sensing block 402. The at least one other component 104 includes a memory 406. For example, the memory 406 can be a non-volatile memory. As a non-limiting example, the memory 406 can be mounted as a portion of a RF chip. The at least one other component 104 also includes a transmitter or transceiver 408. The transmitter or transceiver 408 can be used to transmit data from the apparatus 102 to a handheld device or other computing device (e.g., for further analysis). The example system 400 of FIG. 4 also includes a charge regulator 412. The charge regulator 412 is coupled to the processor unit 414 and memory 416.

A non-limiting example use of system 400 is as follows. An external power source, such as through inductive coupling, provides power for the apparatus 102 to perform the measurements. The processor unit 404 activates, stimulates the analog sensing block 402, which conditions the signal and delivers it to an A/D port on the processor unit 404. The data from apparatus 102 is stored in memory 406. In an example, when a near-field communication (NFC)-enabled handheld device is brought into proximity with the system 400 to provide power through inductive coupling, data is transferred to the handheld device, where it is interpreted by application software of the handheld device. Data transfer can occur.

In a non-limiting example, the system 100, system 150, system 300 or system 400 can be mounted on a backing, such as but not limited to a patch. The backing is disposed over the tissue to be measured.

In a non-limited example, at least a portion of the system 100, system 150, system 300, system 400, or any f the apparatus described herein may be disposed on a substrate. As used in any of the example methods, systems or apparatus described herein, the term “disposed on” is defined to encompass “at least partially embedded in.” The substrate can be formed of any flexible material, such as but not limited to a polymer-based material. In an example, the flexible substrate can be formed from a polydimethylsiloxane (PDMS). In an example, the substrate has a Young's modulus of about 10 GPa or less.

In another non-limited example, at least a portion of the system 100, system 150, system 300, system 400, or any f the apparatus described herein may be covered by an encapsulation layer. The encapsulation layer can be formed from a polymer-based material. For example, the encapsulation layer can be formed from an elastomer, such as but not limited to, a polydimethylsiloxane (PDMS) or a silicone (including SORTACLEAR® silicone, SOLARIS® silicone, or ECOFLEX® silicone (all available from Smooth-On, Inc., Easton, Pa.). In an example, the encapsulation layer has a Young's modulus of about 100 MPa or less. In an example implementation where an apparatus is configured to detect electromagnetic radiation in the visible region of the electromagnetic spectrum, an encapsulation layer formed from a polyimide may be used (since a polyimide can be configured to absorb ultraviolet electromagnetic frequencies).

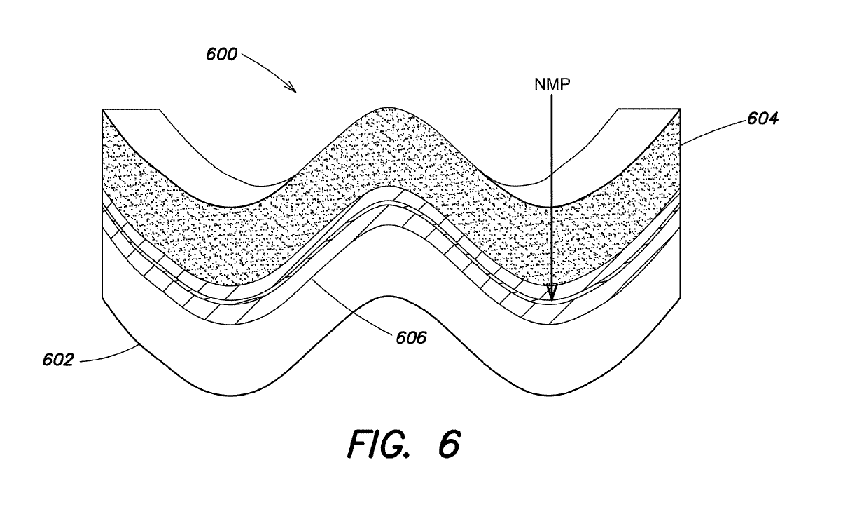

FIG. 5 shows a cross-section of an example apparatus or system according to the principles described herein. The example structure includes a substrate 502, an encapsulation layer 504, and a device layer 506. The device layer 506 includes at least one sensor component 508. In an example, the device layer 506 can include at least one CMOS component 510, such as but not limited to an amplifier, a multiplexer, a data signal filter, or a passive element. In an example, the device layer 506 can include at least one microcontroller and at least one radio component.

In an example, the thickness of the encapsulation layer and substrate can be configured such that a device layer including any of the systems or apparatus according to the principles herein lies at a neutral mechanical plane (NMP) or neutral mechanical surface (NMS) of the system or apparatus. The NMP or NMS lies at the position through the thickness of the device layers for the system or apparatus where any applied strains are minimized or substantially zero. In an example, the NMP or NMS can be positioned at or near a midpoint of a depth of the system or apparatus. The location of the NMP or NMS can be changed relative to the structure of the system or apparatus through introduction of materials that aid in strain isolation in the components of the system or apparatus that are used to perform the electrical measurements of the tissue. For example, the thickness of encapsulating material disposed over the system or apparatus described herein may be modified (i.e., decreased or increased) to depress the system or apparatus relative to the overall system or apparatus thickness, which can vary the position of the NMP or NMS relative to the system or apparatus. In another example, the thickness of the substrate of the apparatus can be used to vary the position of the NMP or NMS relative to the system or apparatus. In another example, the type of encapsulating material, including any differences in the elastic (Young's) modulus of the encapsulating material versus the substrate material, can be used to position the NMP or NMS.

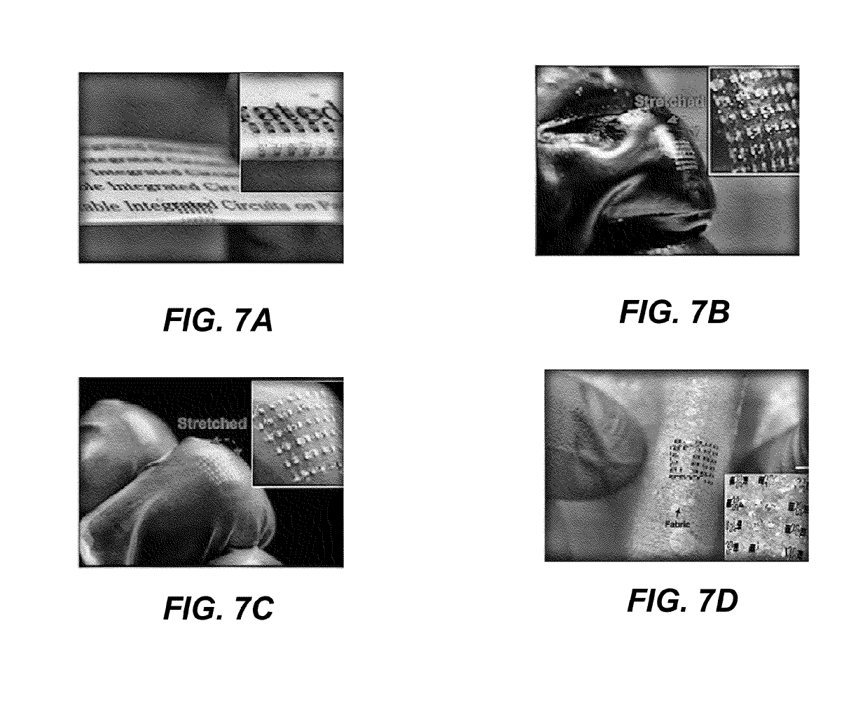

FIG. 6 shows a cross-section of an example layered structure 600 that includes a substrate 602, an encapsulation layer 604, and a device layer 606. The NMP of example structure 600 is indicated by the line going through the structure. As indicated in the FIG. 6, the thickness and type of materials of substrate 602 and encapsulation layer 604 can be chosen such that at least a portion of device layer 606 is positioned at the NMP of the overall structure. In this example, the NMP lies at positioned at or near a midpoint of a depth of the example structure 600.

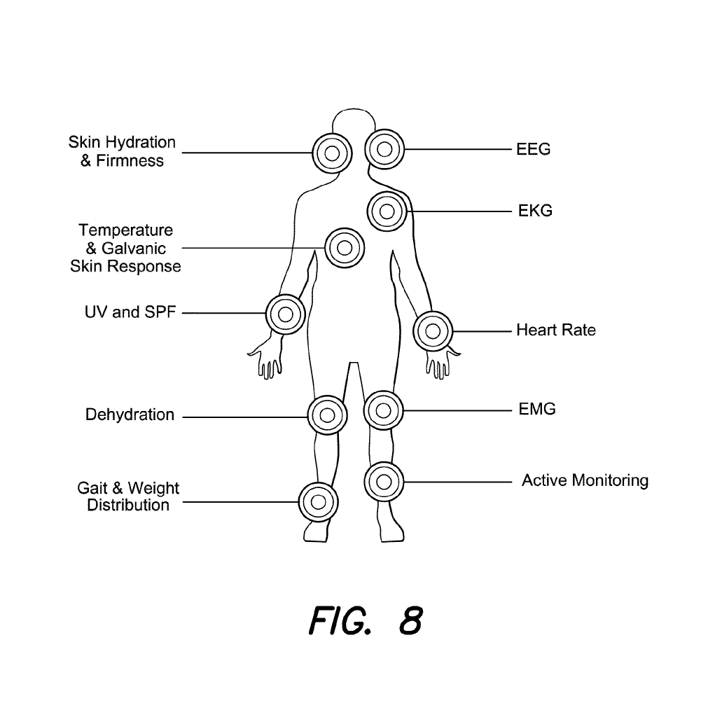

FIGS. 7A-7D shows example apparatus or systems according to the principles herein that are disposed on at least a portion of different types of surfaces. In FIG. 7A, the example apparatus or system is disposed on a portion of the surface of paper. In FIG. 7B, the example apparatus or system is disposed on a portion of the surface of leather. In FIG. 7C, the surface is vinyl, and in FIG. 7D, the surface is a fabric. In an example, the surface that is measured using a sensor component described herein is a surface of fabric such as artwork, vegetation (such as a plant), a tool surface (including other types of equipment), paper, wood, or fabric (including artwork or other works on canvas).

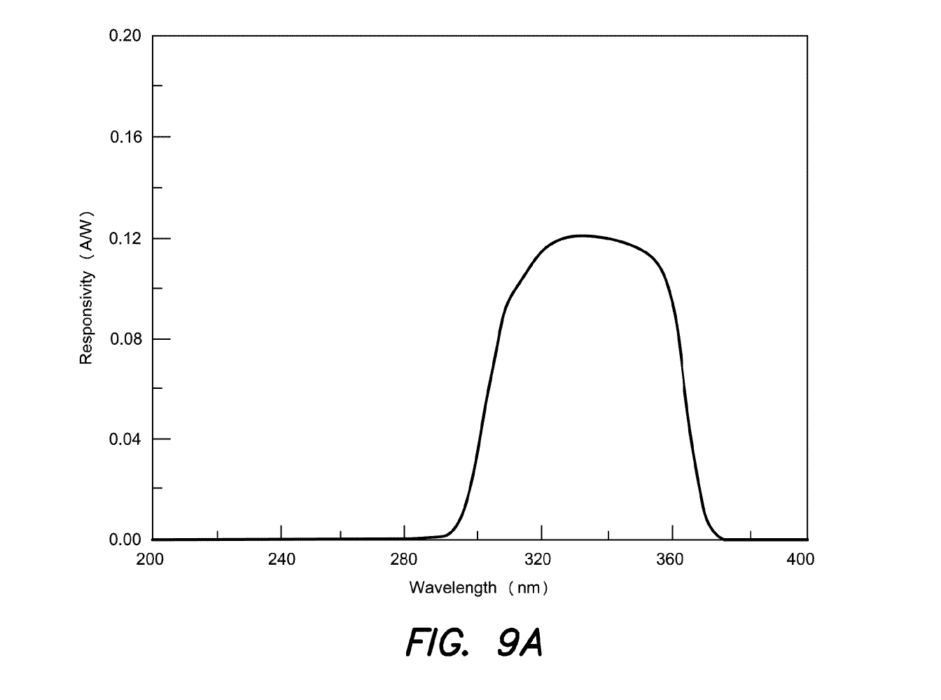

An apparatus or system according to the principles described herein can be used to monitor tissue condition in conjunction with a wide range of other on-body sensors. Non-limiting examples of tissue conditions that may be monitored using one or more of the apparatus described herein are shown in FIG. 8. For example, an apparatus or system herein can include at least one sensor component according to the principles herein for measuring an amount of visible or UV light exposure of the tissue, or an amount of sun protection factor (SPF) provided by a product applied to the tissue. As yet another example, an apparatus herein can be configured to include at least one hydration sensor for measuring a hydration level of the tissue. As another example, an apparatus herein can be configured to include at least one temperature sensor for measuring the temperature of the tissue.

The apparatus and systems of the technology platform described herein support conformal electronics that can be used to log sensor data at very low power levels over extended periods, while providing wireless communication with external computing devices (including handheld devices). The conformal electronics include on-body electronics and electronics that conform to other surfaces (including paper, wood, leather, fabric (including artwork or other works on canvas), a plant or a tool).

The technology platform described herein supports conformal electronics that can be used to monitor an amount of electromagnetic radiation that a surface is exposed to. In an example, the sensor components are UV sensor that allow the continuous recording of UVA and UVB exposure. In a non-limiting example, a system or apparatus described herein can be configured as a visible/UV sensor that records the amount of electromagnetic radiation that a surface is exposed to, and transmits the data measurement to an external computing device (including a handheld device).

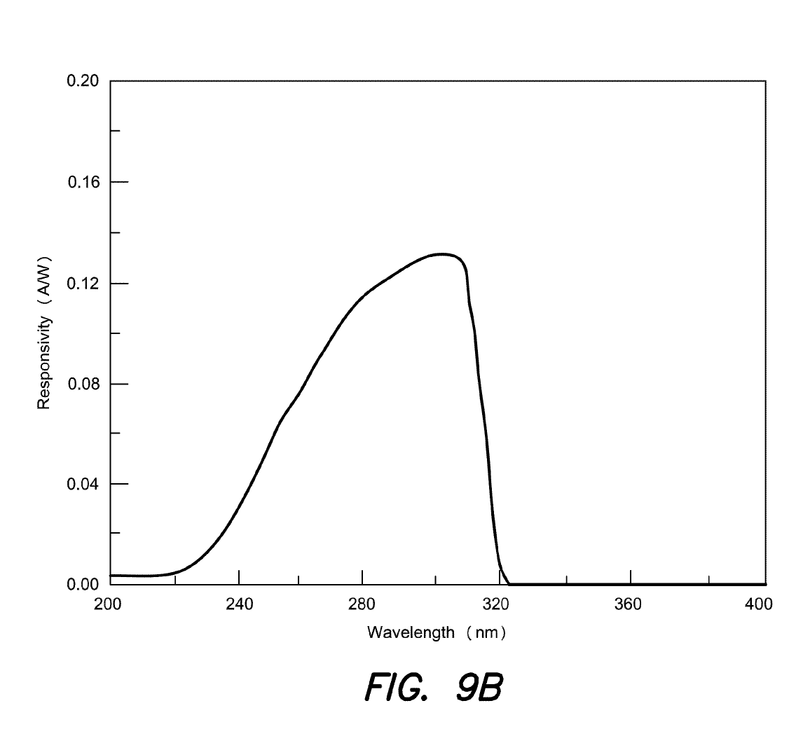

FIGS. 9A and 9B show the wavelength regions of response of UV sensors according to the principles herein when exposed to sunlight. FIG. 9A shows the response for a sensor component configured to respond to UVA wavelengths (from roughly 400 to roughly 280 nm). FIG. 9B shows the response for a sensor component configured to respond to UVB wavelengths (from roughly 325 to roughly 220 nm).

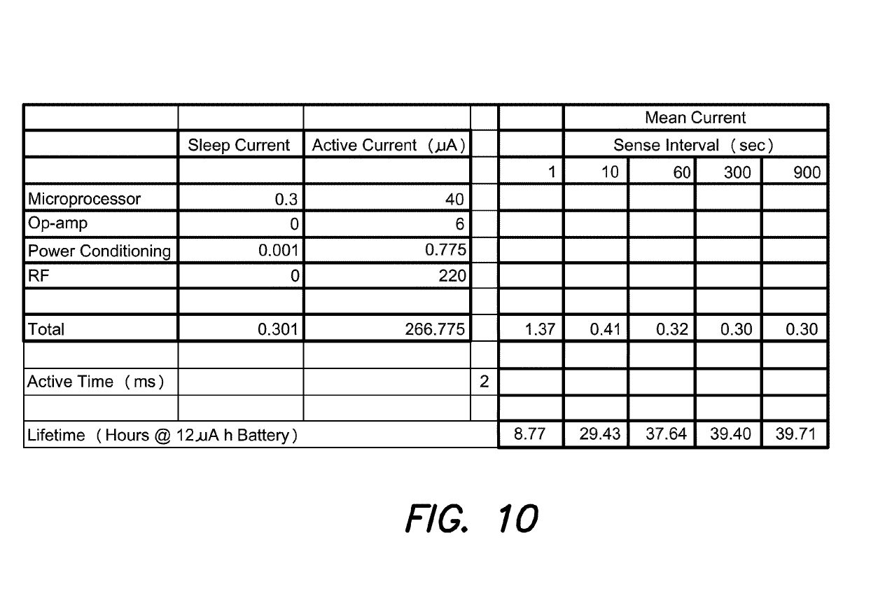

The table in FIG. 10 shows non-limiting example values for sleep current, active current, and mean current (in units of (μA)) from an operation of an apparatus or system according to this example. The table shows a power budget for the system as a function of time for different intervals of sample time. In this example system, the operational amplifiers (op-amps) and RF chip 12C interface can be shut down between read operations, thereby taking no standby power. Based on these results, a 12 μAh battery (such as available from Cymbet Corporation), with a bare die footprint of 2.8×3.5 mm, can be used to support over a day of operation, depending on length of sampling interval.

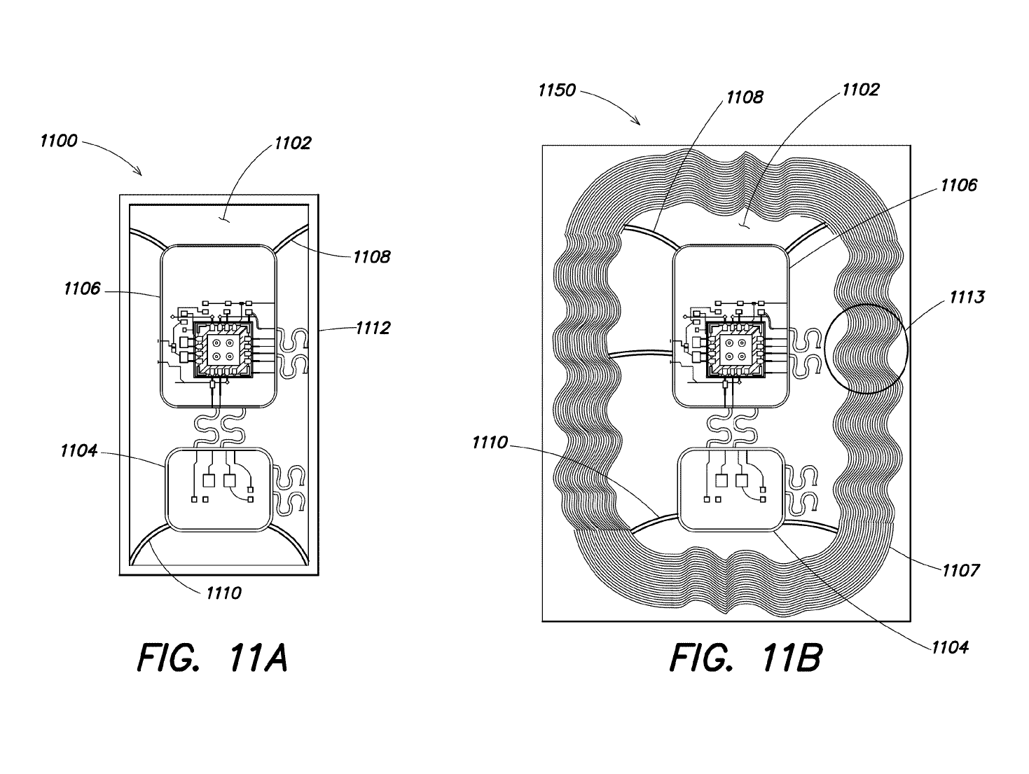

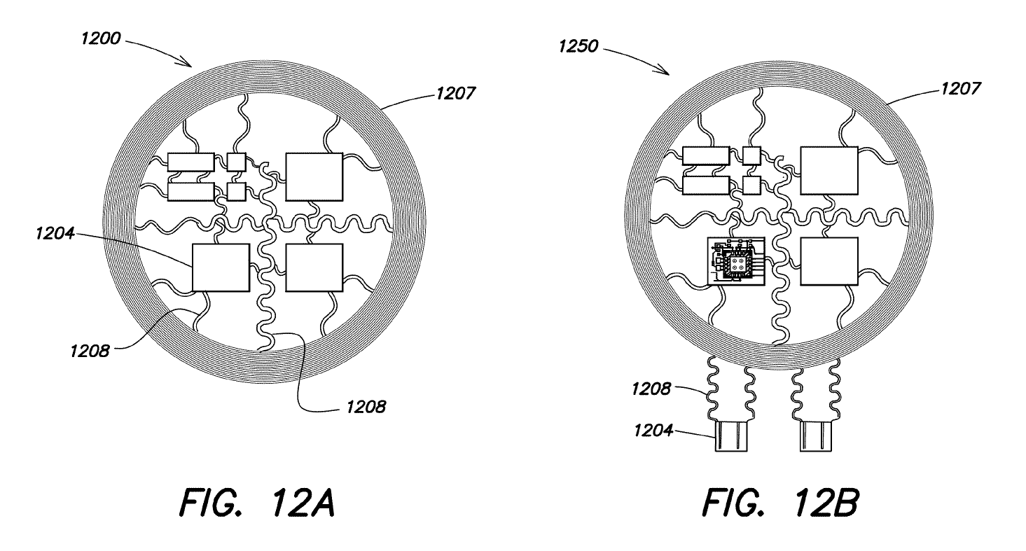

FIG. 11A shows non-limiting example apparatus 1100 according to the principles described herein. The apparatus 1100 includes a flexible substrate 1102, a sensor component 1104 disposed on the flexible substrate and a processing unit 1106 in communication with the sensor component. The sensor component 1104 measures an amount of electromagnetic radiation incident on its exposed surface, where the electromagnetic radiation has frequencies in the visible or ultraviolet regions of the electromagnetic spectrum. As shown in FIG. 11A, apparatus 1100 also includes at least one cross-link structure 1108 that physically couples to a portion of the processing unit 1106. There is also at least one cross-link structures 1110 that physically couples to a portion of the sensor component 1110. The cross-link structure are formed from a dielectric material. The apparatus 1100 can be disposed on a surface of a tissue, an object or an item to be monitored. For example, the surface to be monitored can be a portion of paper, wood, leather, fabric (including artwork or other works on canvas), a plant or a tool. A measure of the amount of electromagnetic radiation incident on the sensor component can be used to provide an indication of an amount of exposure of the surface to the electromagnetic radiation.

The flexible substrate 1102 can be formed from a polymer-based material. For example, the substrate can be formed from an elastomer, such as but not limited to PDMS or a silicone-based material. As other non-limiting examples, the flexible substrate 1102 can be formed from a flexible plastic, paper, or fabric. In an example, the flexible substrate has a Young's modulus of less than about 10 GPa.

According to the principles herein, the cross-link structures shown and/or described in any of the apparatus or systems herein are used to provide mechanical stability to the apparatus or system. For example, given that the substrate of the apparatus is flexible (that is, not rigid), the apparatus or system can be subjected to bending, torsion, elongation, compression, deformation, or other such forces during use. These forces can change a form factor of the apparatus or system. In another example, these forces can cause some components of the system or apparatus to be moved out of alignment, which can cause certain electrical interconnects between the components to be weakened or damaged, thereby affecting the performance of the apparatus or system. The cross-link structures described herein are disposed at selected regions of the apparatus or system to provide mechanical stability to the structure against these externally applied forces. For example, one end of a cross-link structure can be physically coupled to a portion of a component of the apparatus or system and the other end can be coupled to another component, or to the substrate.

The cross-link structures according to any of the example systems or apparatus herein also can be formed from a polymer-based material. For example, the cross-link structure can be formed from PDMS, a silicone, or any other applicable elastomer. As another example, the cross-link structure can be formed from a polyimide. In an example, the flexible substrate and the cross-link structure can be formed from the same material. In another example, the flexible substrate and the cross-link structure can be formed from different materials.

In the non-limiting example of FIG. 11A, cross-link structures 1108 and 1110 physically couple a component (1104 or 1106) to a portion of substrate 1102. In another example, cross-link structures may be used to physically couple sensor component 1104 to processing unit 1106.

The apparatus 1100 may include a memory in communication with sensor component 1104 to stores any data from a measurement. For example, the data can be indicative of measurements of the amount of electromagnetic radiation incident on sensor component 1104. In an example, the memory may store machine readable instructions that cause the processing unit 1106 to analyze the measurement data to provide an indication of the amount of exposure of the surface to the electromagnetic radiation.

The apparatus 1100 may include a brace structure formed from a dielectric material to which the cross-link structures may be physically coupled. For example, the brace structure may be formed as a coil or looped structure on the flexible substrate, an end of one or more of the cross-link structures can be physically coupled to it, and the other end of the cross-link structure(s) can be physically coupled to a portion of sensor component 1104 and/or to a portion of processing unit 1106. As an example, feature 1112 of FIG. 11A may be formed as a brace structure (as opposed to being a portion of substrate 1102). The combined action of the brace structure and the cross-link structure(s) may enhance the mechanical stability to the apparatus or system against externally applied forces (as described above).

The brace structure also can be formed from a polymer-based material, such as but not limited to a polyimide, PDMS, a silicone, or other applicable elastomer.

In different examples, the brace structure and the cross-link structure may be formed from the same material or they may be formed from different materials.

FIG. 11B shows another example apparatus 1150 that includes flexible substrate 1102, sensor component 1104 disposed on the flexible substrate 1102, a processing unit 1106 in communication with the sensor component 1104, and a coil structure 1107 disposed on the substrate. Coil structure 1107 is formed from a conductive material and can be used as an antenna. Coil structure 1107 has a rectangular shape in this example. However, coil structure 1107 can be polygonal-shaped, circular-shaped, square-shaped or any other geometric shape.

In any example apparatus, method or system described herein, the coil structure can be formed from a metal, such as but not limited to, Al or a transition metal (including Au, Ag, Cr, Cu, Fe, Ir, Mo, Nb, Pd, Pt, Rh, Ta, Ti, V, W or Zn), or any combination thereof, or a doped semiconductors, including any conductive form of Si, Ge, or a Group III-IV semiconductor (including GaAs, InP).

As shown in FIG. 11B, coil 1107 includes at least one corrugated portion 1112. For example, the corrugated portion can have a zig-zag-shaped, serpentine-shaped, grooved-shaped, or rippled structure.

In the example of FIG. 11B, the sensor component 1104 and the processing unit 1106 are surrounded by coil structure 1107. In another example, either the sensor component 1104 or the processing unit 1106 may be positioned outside of the coil structure 1107. Any description above in connection with the components or features of FIG. 11A are also applicable to the equivalent features or components of FIG. 11B. In an example, the cross-link structures 1108, 1110 may link to portions of the coil structure 1107 that are closer to the center. In another example, the cross-link structures 1108, 1110 may link to the outer portions of the coil structure 1107.

In various example implementations, coil structure 1107 can be used to transmit a RF signal from the apparatus 1150 to an external device or can be used to receive a signal from the device external. For example, apparatus 1150 may also include a radio-frequency component in communication with the coil structure 1107 and/or the processing unit 1106. The radio-frequency component can use the coil structure 1107 to transmit the measured amount of electromagnetic radiation incident on the sensor component 1104 and/or the indication of the amount of exposure of the surface (on which the apparatus 1150 is disposed) to the electromagnetic radiation. In an example, the radio-frequency component can be a BLUETOOTH® component (Bluetooth SIG, Kirkland, Wash.).

In a non-limited example, at least a portion of apparatus 1150 is covered by an encapsulation layer. The encapsulation layer can be formed from a polymer-based material. For example, the encapsulation layer can be formed from an elastomer, such as but not limited to, a polydimethylsiloxane (PDMS) or a silicone (including SORTACLEAR® silicone, SOLARIS® silicone, or ECOFLEX® silicone (all available from Smooth-On, Inc., Easton, Pa.). In an example, the encapsulation layer has a Young's modulus of about 100 MPa or less. In an example implementation where an apparatus is configured to detect electromagnetic radiation in the visible region of the electromagnetic spectrum, an encapsulation layer formed from a polyimide may be used (since a polyimide can be configured to absorb ultraviolet electromagnetic frequencies). As described above, the thickness of the encapsulation layer and flexible substrate 1102 and type of materials used for the encapsulation layer and flexible substrate 1102 can be selected such that the sensor component 1104 and the processor unit are positioned at or near a midpoint of a depth of the apparatus (i.e., near a NMP).

In an example, portions of the flexible substrate can include an adhesive. The adhesive can be used to attach the portions of the flexible substrate to the surface.

Sensor component 1104 may include a photodetector. Non-limiting examples of applicable photodetectors include a silicon-based photodetector, a silicon carbide-based photodetector, a germanium-based photodetector, a gallium nitride-based photodetector, an indium gallium nitride-based photodetector and an aluminum gallium nitride-based photodetector. In an example, sensor component 1104 may be a photodetector that includes one or more p-n junctions.

The apparatus 1100 or 1150 may include at least one filter that is disposed above sensor component 1104 in the areas exposed to the electromagnetic radiation. A measure of the electromagnetic radiation using the filter(s) and the at least one sensor component can be used to provide a measure of the amount of ultraviolet-A electromagnetic radiation and/or ultraviolet-B electromagnetic radiation incident on the surface.

In an example implementation, the apparatus 1100 or 1150 can include two sensor component, wherein one of the sensor components is stacked over the other sensor component to provide a stacked sensor component. In this example, a comparison of a measure of the electromagnetic radiation using the stacked sensor component to a measure of the electromagnetic radiation using another of the at least one sensor components can be used to provide a measure of the amount of ultraviolet-A electromagnetic radiation and/or ultraviolet-B electromagnetic radiation incident on the surface.

The measure of the amount of exposure of the surface of a surface to the electromagnetic radiation can be used to provide a measure of a level of SPF protection of the surface (e.g., of a product applied to the surface). For example, a comparison of a measurement of the electromagnetic radiation made using a sensor component that includes an ultraviolet filter to a measurement of the electromagnetic radiation using another of the at least one sensor components having no ultraviolet filter can be used to provide the measure of a level of SPF protection of the tissue.

In a non-limiting example, the apparatus 1150 can include an amplifier in electrical communication with the at least one sensor component. The amplifier can be used to amplify the signal from the measurement of the sensor component 1104 before it is analyzed by the processor unit 1106.

An example system for monitoring exposure of a surface to electromagnetic radiation is also provided. The example system includes an apparatus according to any of the principles described herein and a reader device. The reader device can be used to receive from the apparatus data indicative of the measure of the amount of electromagnetic radiation incident on the sensor component and/or the indication of the amount of exposure of the surface to the electromagnetic radiation. The surface can be a portion of paper, wood, leather, fabric (including artwork or other works on canvas), a plant or a tool

In an example, the reader can include a coupling member. When the coupling member is electrically coupled to a portion of the apparatus, the reader device receives the data indicative of the measure of the amount of electromagnetic radiation incident on the at least one sensor component and/or the indication of an amount of exposure of the surface to the electromagnetic radiation.

The reader device can be a near-field communication (NFC)-enabled handheld device. In an example, when a near-field communication (NFC)-enabled handheld device is brought into proximity with the system 150, data is transferred to the handheld device, where it is interpreted by application software of the handheld device. In another example, the data can be analyzed using the processor of the apparatus, and the result of the analysis can be transferred to the handheld device, such as the indication of the amount of exposure of the surface to the electromagnetic radiation or a value of an SPF protection from a product applied to the surface.

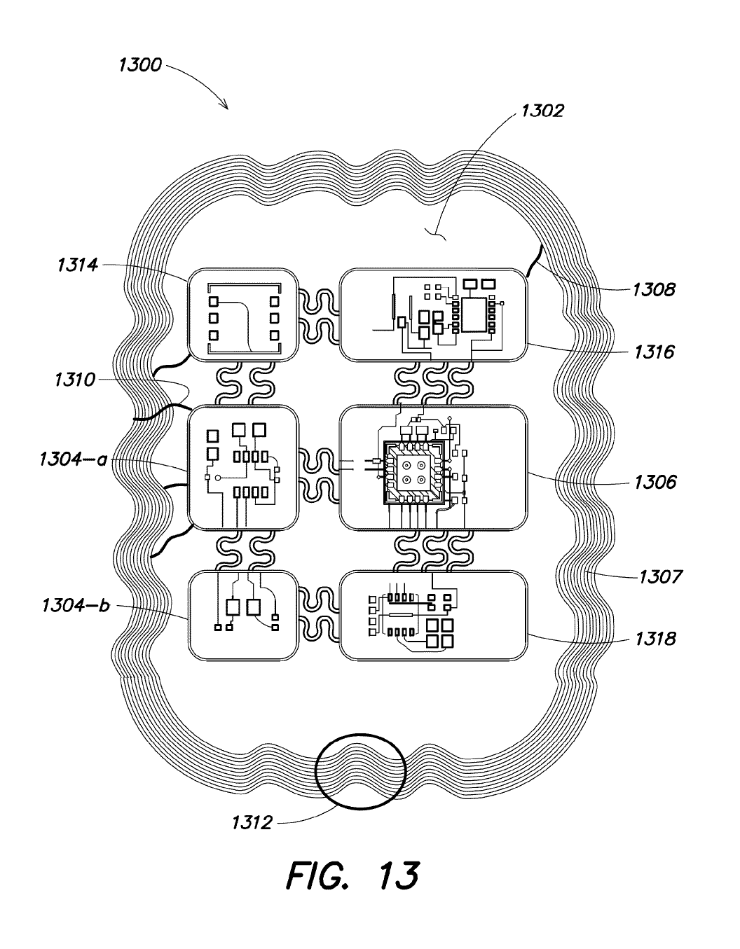

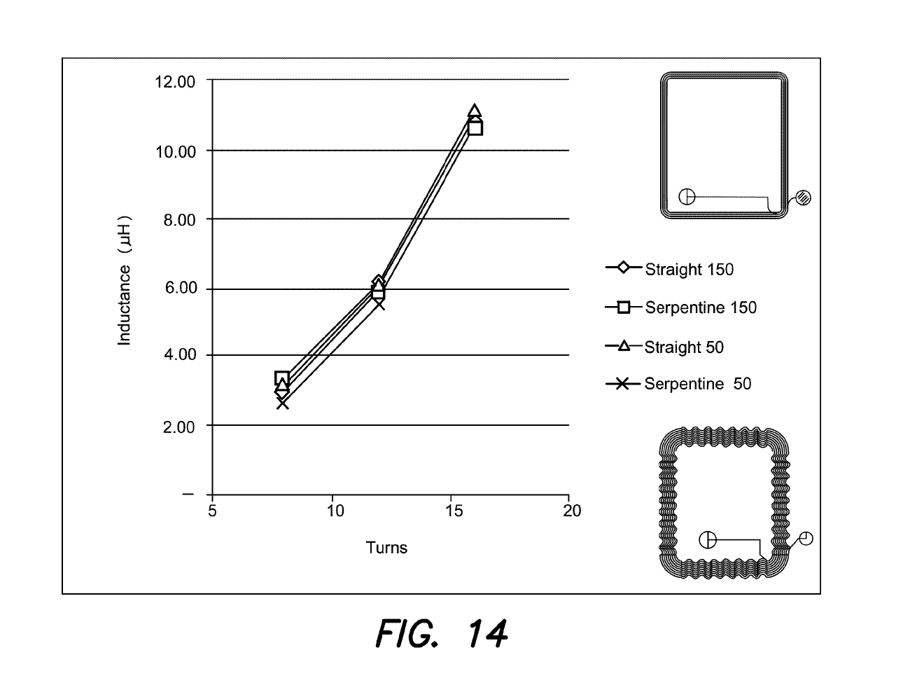

FIG. 12A shows another example apparatus 1200 that includes sensor component 1204, coil structure 1207, and cross-link structures 1208. Any description above in connection with the components or features of FIG. 11A or 11B are also applicable to the equivalent features or components of FIG. 12A. As shown in FIG. 12A, cross-link structure physically couples a portion of the sensor component 1204 to a portion of the coil structure 1207. The cross-link structures are formed from a flexible material that is non-conductive. In an example, the cross-link structures 1208, 1210 may link to portions of the coil structure 1207 that are closer to the center. In another example, the cross-link structures 1208, 1210 may link to the outer portions of the coil structure 1207. The measure of the amount of electromagnetic radiation incident on the sensor component 1204 provides an indication of the amount of exposure of the surface to the electromagnetic radiation.

In the example of FIG. 12A, the coil structure 1207 surrounds sensor component 1204. In another example, the sensor component 1204 may be positioned outside of the coil structure 1207. FIG. 12B shows another example apparatus 1250 that includes sensor component 1204, coil structure 1207, and cross-link structures 1208. In this example, the sensor 1204 is positioned outside the coil structure 1207.

In different examples, the sensor component 1204 can include a photodetector, a hydration sensor, a temperature structure, or any type of sensor.

Coil structure 1207 (shown in FIGS. 12A and 12B) is formed from a conductive material and can be used as an antenna. In these examples, coil structure 1207 has a circular shape. However, coil structure 1207 can be polygonal-shaped, square-shaped, rectangular-shaped, or any other geometric shape. In an example, coil 1207 can include corrugated portions, including portions having a zig-zag shape, a serpentine shape, a grooved shape, or a rippled structure.

Example apparatus 1200 or 1250 can include a processing unit. In an example, the processing unit can be used to analyze the measure of the amount of electromagnetic radiation incident on the sensor component 1204 to provide the indication of the amount of exposure of the surface to the electromagnetic radiation. In this example, apparatus 1200 or 1250 can include a radio-frequency component in communication with the coil structure 1207 and the processing unit. The radio-frequency component can be used to transmit the measure of the amount of electromagnetic radiation incident on the at least one sensor component and/or the indication of an amount of exposure of the surface to the electromagnetic radiation using the at least one coil structure.

In another example, example apparatus 1200 or 1250 can include a flexible substrate, where sensor component 1204 and coil structure 1207 are disposed on the flexible substrate. The flexible substrate can be a polymer, as described in connection with FIG. 11A or 11B. In different examples, the flexible substrate and the cross-link structure can be formed from the same material or from different materials. In an example, portions of the flexible substrate can include an adhesive. The adhesive can be used to attach the portions of the flexible substrate to the surface.

Coil structure 1107 can be used to transmit a RF signal from the example apparatus 1200 or 1250 to an external device or can be used to receive a signal from the device external. For example, example apparatus 1200 or 1250 may also include a radio-frequency component in communication with the coil structure 1207. The radio-frequency component can use the coil structure 1207 to transmit the measured amount of electromagnetic radiation incident on the sensor component 1104 and/or the indication of the amount of exposure of the surface (on which the apparatus 1250 is disposed) to the electromagnetic radiation. In an example, the radio-frequency component can be a BLUETOOTH® component (Bluetooth SIG, Kirkland, Wash.).

In a non-limited example, at least a portion of example apparatus 1200 or 1250 is covered by an encapsulation layer. The encapsulation layer can be formed from a polymer-based material as described above. Where an apparatus is configured to detect electromagnetic radiation in the visible region of the electromagnetic spectrum, an encapsulation layer formed from a polyimide may be used. As described above, the example apparatus 1200 or 1250 can be configured such that the sensor component 1204 is positioned at or near a midpoint of a depth of example apparatus 1200 or 1250 (i.e., near a NMP).

Sensor component 1204 may include a photodetector. Non-limiting examples of applicable photodetectors include a silicon-based photodetector, a silicon carbide-based photodetector, a germanium-based photodetector, a gallium nitride-based photodetector, an indium gallium nitride-based photodetector and an aluminum gallium nitride-based photodetector. In an example, sensor component 1204 may be a photodetector that includes one or more p-n junctions.

The apparatus 1200 or 1250 may include at least one filter that is disposed above sensor component 1104 in the areas exposed to the electromagnetic radiation. A measure of the electromagnetic radiation using the filter(s) and the at least one sensor component can be used to provide a measure of the amount of ultraviolet-A electromagnetic radiation and/or ultraviolet-B electromagnetic radiation incident on the surface.

In an example implementation, the apparatus 1200 or 1250 can include two sensor component, wherein one of the sensor components is stacked over the other sensor component to provide a stacked sensor component. In this example, a comparison of a measure of the electromagnetic radiation using the stacked sensor component to a measure of the electromagnetic radiation using another of the at least one sensor components can be used to provide a measure of the amount of ultraviolet A electromagnetic radiation and/or ultraviolet B electromagnetic radiation incident on the surface.

The measure of the amount of exposure of the surface of a surface to the electromagnetic radiation can be used to provide a measure of a level of SPF protection of the surface (e.g., of a product applied to the surface). For example, a comparison of a measurement of the electromagnetic radiation made using a sensor component that includes an ultraviolet filter to a measurement of the electromagnetic radiation using another of the at least one sensor components having no ultraviolet filter can be used to provide the measure of a level of SPF protection of the tissue.

In a non-limiting example, the example apparatus 1200 or 1250 can include an amplifier in electrical communication with the at least one sensor component. The amplifier can be used to amplify the signal from the measurement of the sensor component 1204 before it is analyzed by the processor unit 1206.

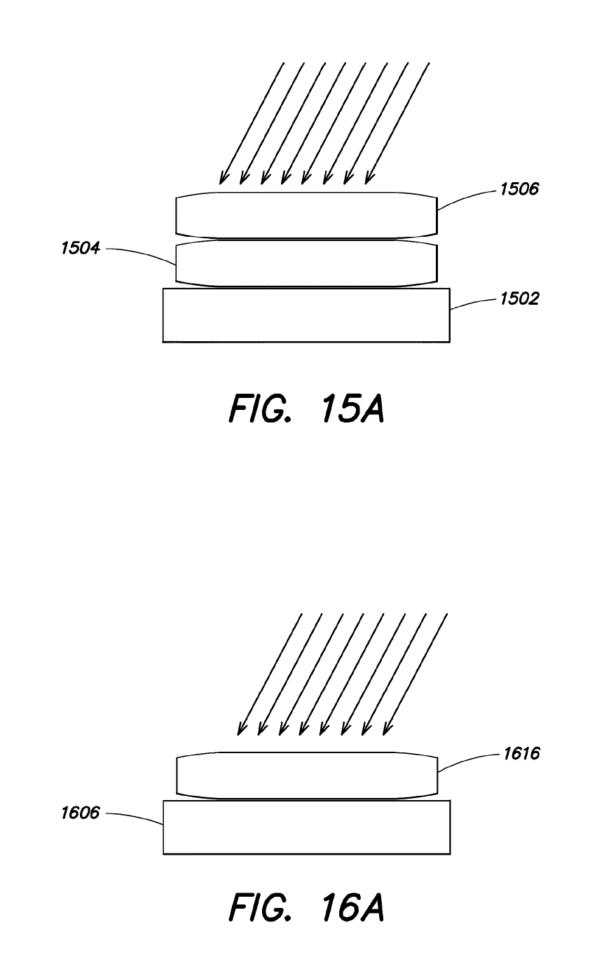

FIG. 13 shows an example apparatus 1300 that includes flexible substrate 1302, two sensor components (1304-a and 1304-b) disposed on the flexible substrate 1302, a processing unit 1306 in communication with the sensor component 1304, and a coil structure 1307 disposed on the substrate. Coil structure 1307 is formed from a conductive material and can be used as an antenna. Coil structure 1307 can be polygonal-shaped, circular-shaped, square-shaped or rectangular-shaped.

As shown in FIG. 13, coil 1307 includes corrugated portions 1312. For example, the corrugated portion can have a zig-zag-shaped, serpentine-shaped, grooved-shaped, or rippled structure.

In this example, the sensor component 1304 and the processing unit 1306 are surrounded by coil structure 1307. In another example, either the sensor component 1304 or the processing unit 1306 may be positioned outside of the coil structure 1307. Any description above in connection with the components or features of FIG. 11A, 11B, 12A or 12B are also applicable to the equivalent features or components of FIG. 13. In an example, the cross-link structures 1308, 1310 may link to portions of the coil structure 1307 that are closer to the center. In another example, the cross-link structures 1308, 1310 may link to the outer portions of the coil structure 1307.

In this example, apparatus 1300 also includes a battery 1314, a charging regulator 1316, and a RF component 1318. As shown in FIG. 13, electrical interconnect structures electrically connect the RF component with the processing unit 1306. Battery 1314 provides power to the various components of apparatus 1300. Coil structure 1307 is used to transmit a RF signal from the apparatus 1200 to an external device and/or to receive a signal from the device external. The radio-frequency component can use the coil structure 1307 to transmit the measured amount of electromagnetic radiation incident on the sensor component 1304 and/or the indication of the amount of exposure of the surface (on which the apparatus 1300 is disposed) to the electromagnetic radiation.

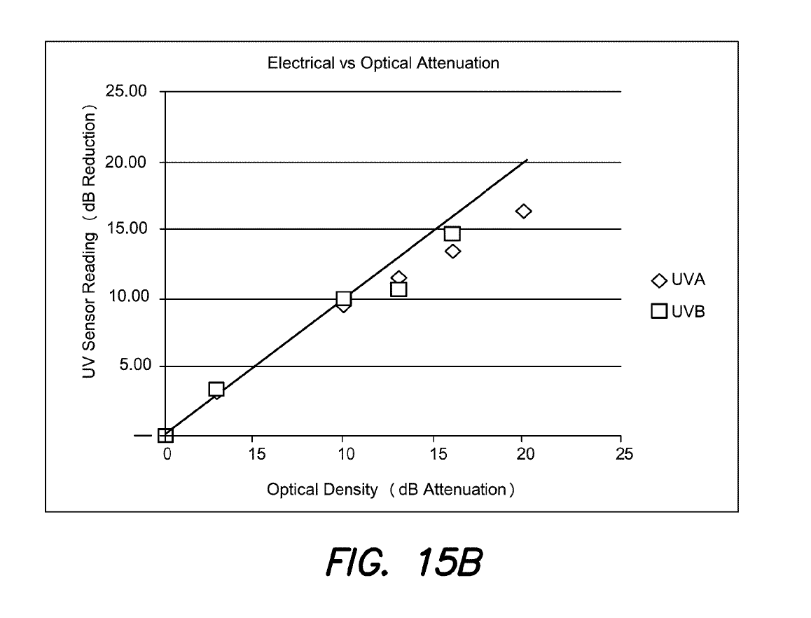

As described above, the coil structures described herein can be used as antenna structures. The corrugated portions of the coil structure allow the apparatus to be stretch with out adversely affecting the inductance properties of the coil. FIG. 14 shows measurement of the inductance (in units of microHenry (μH)) for a rectangular-shaped coil that does not include corrugated portions and a rectangular-shaped coil that includes corrugated portions versus the number of turns of the coils. As shown in FIG. 14, the inductance of the corrugated coil does not change appreciably from that for the straight coil.

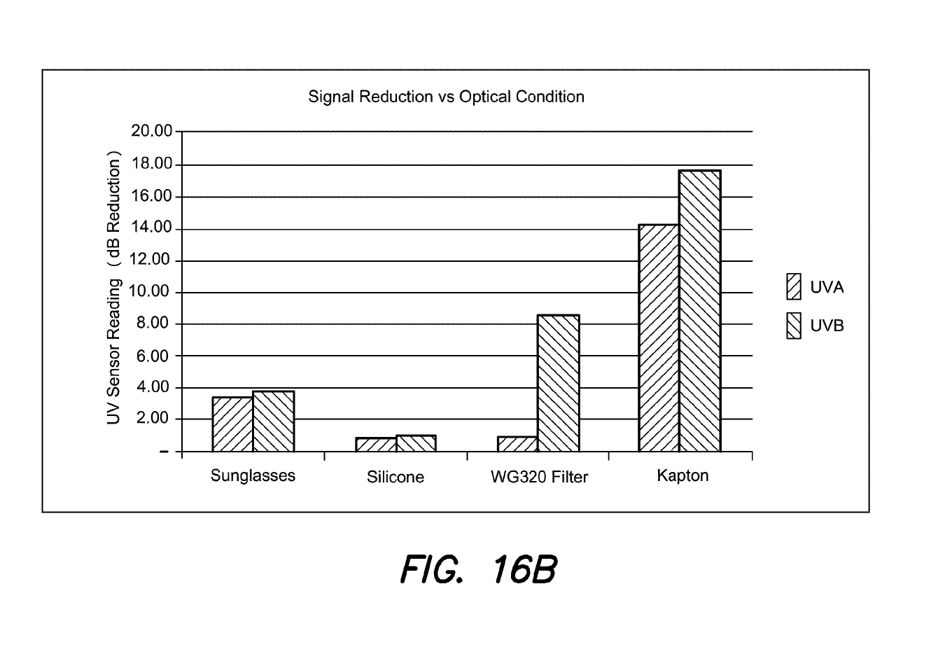

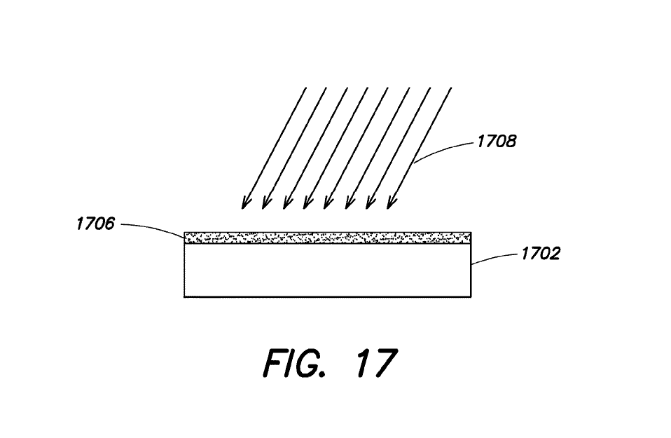

FIGS. 15A and 15B show an example implementation of a method for calibrating a measurement of a sensor component. Measurements can be made using a sensor component that has at least one filter in the path between the sensor component and the electromagnetic radiation. In the example of FIG. 15A, measurements are made using a sensor component 1504 that has two filters 1504, 1506 positioned in the path between the sensor component and the electromagnetic radiation. Multiple combinations of OD0.3, OD1 filters can be used. FIG. 16B shows a plot of the electrical versus optical attenuation in the structures Direct and linear correlation between optical power on sensor and electrical output is observed.

FIGS. 16A and 16B show an example implementation of a method for measurement of different UV blockers using the sensor components described herein. Measurements can be made using a sensor component that has at least one UV blocker in the path between the sensor component and the electromagnetic radiation. The plot of FIG. 16B shows the values of the measurement of UVA and UVB blocking capability of sunglasses, silicone, a WG320 filter and KAPTON® (DuPont, Wilmington, Del.). The results indicate that silicone encapsulation can be transmissive, while KAPTON® is strongly blocking. The WG320 filter is observed to discriminate UVB vs. UVA. Sunglasses are equivalent to a SPF of 2.2.

Photodetection Sensors

As discussed above in connection with any of the example apparatus, systems or methods, the sensor component can be a photodetector.

A number of apparatus, systems, and methods herein use optical detection. Non-limiting example applications include UV sensing for sun protection, infra red (IR) detection to support medical applications in the “therapeutic window”, IR detection to allow input via remote controls (such as for TV), and response to room lighting.

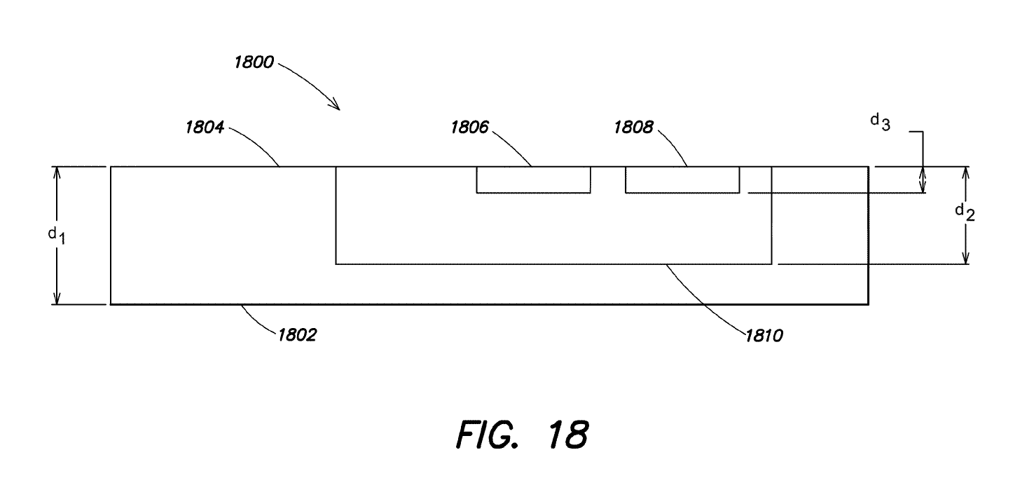

FIG. 17 shows an example photodetector 1702 that can be used as a sensor component in any of the systems, methods and apparatus described herein. The photodetector 1702 is formed from a photosensitive substrate. In a non-limiting example, a change in a measured electrical property of the substrate can be used to provide a measure of the amount of electromagnetic radiation 1708 that the photodetector 1702 is exposed to. A filter 1706 may be used with the photodetector 1702 to selectively exclude electromagnetic radiation that is outside the wavelength range of interest.

A conformal system for such sensing applications can be constructed based on stretchy CMOS. In non-limiting examples, the photodetector may be formed based on a silicon, a silicon carbide, a germanium, a gallium nitride, an indium gallium nitride, or an aluminum gallium nitride substrate.

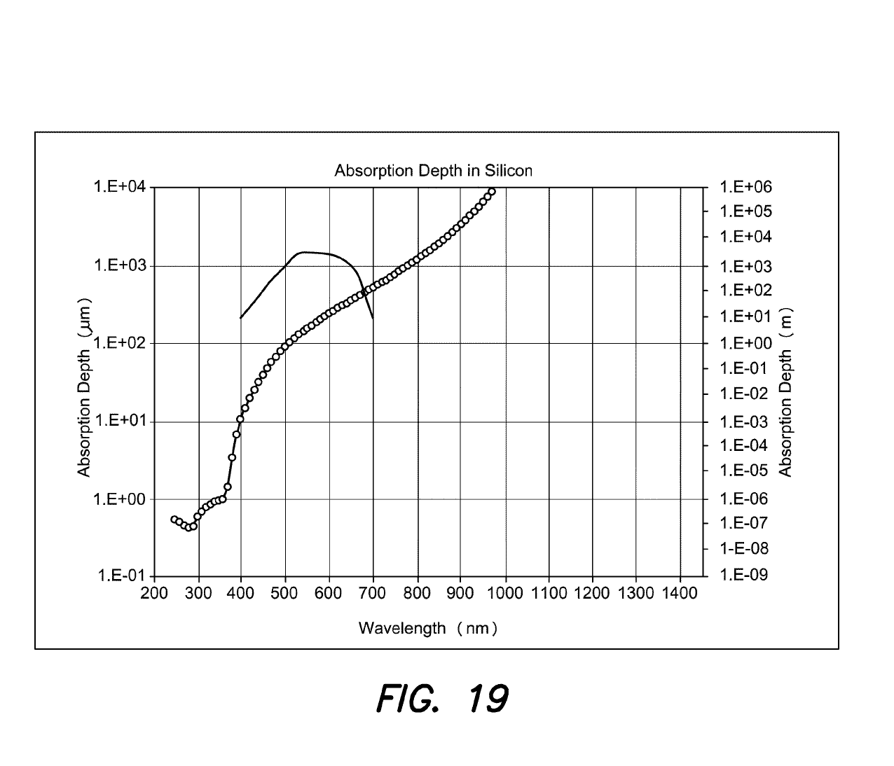

FIG. 18 shows a non-limiting example photodetector 1800 according to the principles herein. Photodetector 1800 can be incorporated into any of the sensor components, apparatus, or systems described herein and be used for detecting electromagnetic radiation. Example photodetector 1800 is formed in a substrate 1802. Substrate 1802 has a surface 1804 that is exposed to electromagnetic radiation. Photodetector 1800 includes an electron collector region 1806 and a hole collector region 1808 disposed in the substrate. A potential well region 1810 is disposed in the substrate and surrounds at least a portion of the electron collector region 1806 and at least a portion of the hole collector region 1808. A portion of the potential well region 1810 is disposed between the electron collector region 1806 and the hole collector region 1808.

The electron collector region 1806 can be positioned proximate to the surface of the substrate 1802 or can be embedded in the substrate 1802. The hole collector region 1808 can be positioned proximate to the surface of the substrate 1802 or can be embedded in the substrate 1802.

As non-limiting examples, substrate 1802 can be formed from a silicon, a silicon carbide, a germanium, a gallium nitride, an indium gallium nitride, or an aluminum gallium nitride material.

The electron collector region 1806 is formed from a n+-type material (i.e., highly-donor-doped semiconductor material). The hole collector region 1808 is formed from a p+-type material (i.e., highly-acceptor-doped semiconductor material).

The potential well region 1810 can be formed from a donor doped semiconductor material (n-type material) if the substrate 1802 is a p-type semiconductor material. If the substrate 1802 is a n-type semiconductor material, the potential well region 1810 can be formed from an acceptor doped semiconductor material (p-type material).

In an example where the potential well region 1810 is formed from a donor doped semiconductor material and the substrate 1802 is a p-type semiconductor material, the potential well region 1810 has a lower concentration of dopants than the electron collector region 1806. In an example where the potential well region 1810 is formed from an acceptor doped semiconductor material and the substrate 1802 is a n-type semiconductor material, the potential well region 1810 has a lower concentration of dopants than the hole collector region 1806.

The substrate 1802 can have a thickness (d₁) of about 10 microns (μm), about 5 microns, about 3 microns, about 2 microns, about 1 micron, or less than about 1 micron.

The potential well region 1810 can have a thickness (d₂) less than or approximately equal to the thickness of the substrate 1802. For example, the potential well region 1801 can have a thickness (d₂) less than about 1 micron, about 1 micron, about 3 microns, about 4 microns, or greater than about 4 microns.

The electron collector region 1806 or the hole collector region 1808 can have a thickness (d₃) less than or approximately equal to the thickness of the potential well region 1810. For example, the electron collector region 1806 or the hole collector region 1808 can have a thickness (d₂) less than about 1 micron, about 1 micron, about 2 microns, about 3 microns, or greater than about 3 microns.

When an incoming photon (electromagnetic radiation) is absorbed, it excites an electron-hole pair. The electron collector region 1806 and the hole collector region 1808 help to separate the holes from the electrons, providing photo-sensing activity. Any change in the carrier concentration, and hence the electrical properties, in the electron collector region 1806 and/or the hole collector region 1808 can be quantified as a measure of the amount of electromagnetic radiation that was absorbed. The amount of electromagnetic radiation that the photodetector is exposed to can be quantified based on the measure of the amount of electromagnetic radiation absorbed in the electron collector region 1806 and/or the hole collector region 1808. The electron collector region 1806 and the hole collector region 1808 can be so heavily doped that photo-carriers generated inside them (based on photon absorption) recombines before they can be collected, and hence not quantified. Photo-carriers generated in the potential well region 1810 are collected in the hole collector region 1808 (for holes as carriers) or the electron collector region 1806 (for electrons as carriers).

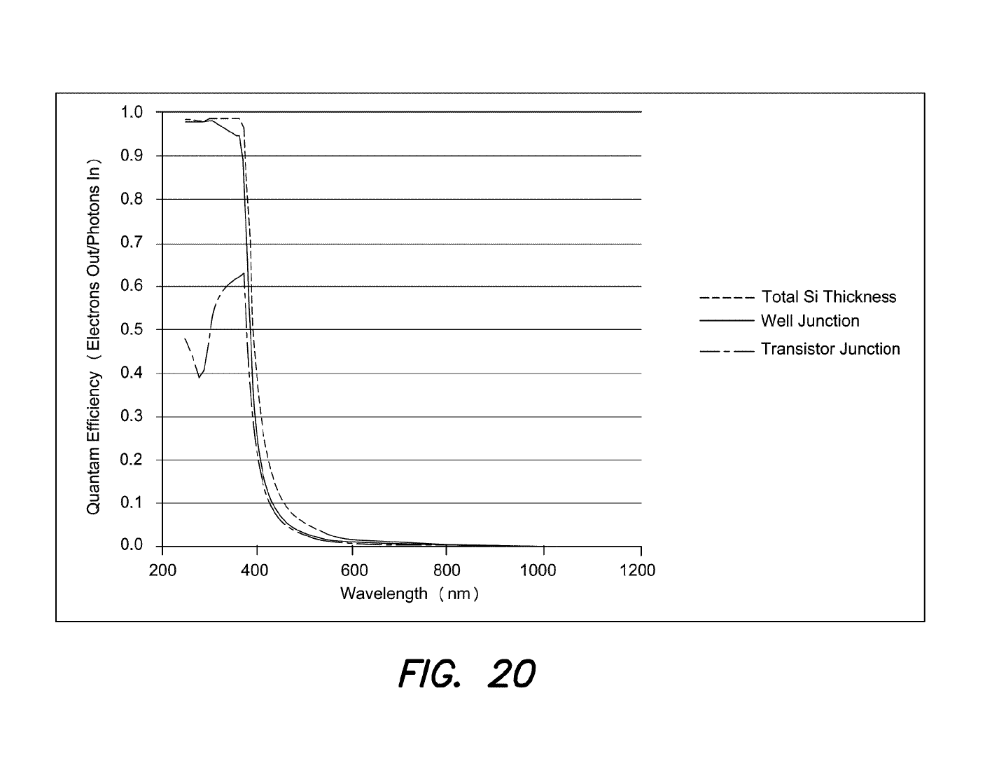

In a non-limiting example, the photodetector can be produced based on silicon. FIG. 19 shows the absorption depth of silicon over a wide range of wavelengths. The vertical axis is the absorption depth, i.e., a measure of the depth over which about 1/e of the incoming electromagnetic radiation energy is absorbed. About 85% of the energy is absorbed in two absorption depths, and only 5% goes beyond three absorption depths. The curve of FIG. 18 also can be used to estimate of the thickness of silicon for absorbing about 30% of the incoming energy, or about half the thickness for absorbing about 85% of the energy. As shown in FIG. 18, longer wavelengths of electromagnetic radiation have a longer absorption depth, until a wavelength (λ) of about 300 nm (at which there is a slight change in the behavior of the UV absorption curve. A silicon layer of over 1 cm thickness is needed to absorb appreciable amounts of electromagnetic radiation of around 1000 nm wavelength. The response of the human eye is represented schematically by the shorter curve ranging from 400 nm to 700 nm, to indicate the visible region of the electromagnetic spectrum.

The quantum efficiency (QE) of the potential well region 1810 to first order can be expressed as 1−e^{-X}^{_{w}}^{/d(λ)}, where d(λ) is the wavelength dependent absorption depth (specific to different materials, such as silicon in FIG. 19), and Xw is the well depth. The bulk of the substrate may also serve as a photo-detector, if surface losses are not too high. The QE of the total thickness of the substrate, where the carriers are collected through lateral collection to the collector regions, is 1−e^{-X}^{_{Si}}^{/d(λ)} where X_{Si} is the substrate thickness. The QE of the electron collector region 1806 and the hole collector region 1808 is e^{-X}^{_{J}}^{/d(λ)}−e^{-X}^{_{W}}^{/d(λ)} where X_{J} is the depth of the electron collector region 1806 and the hole collector region 1808.

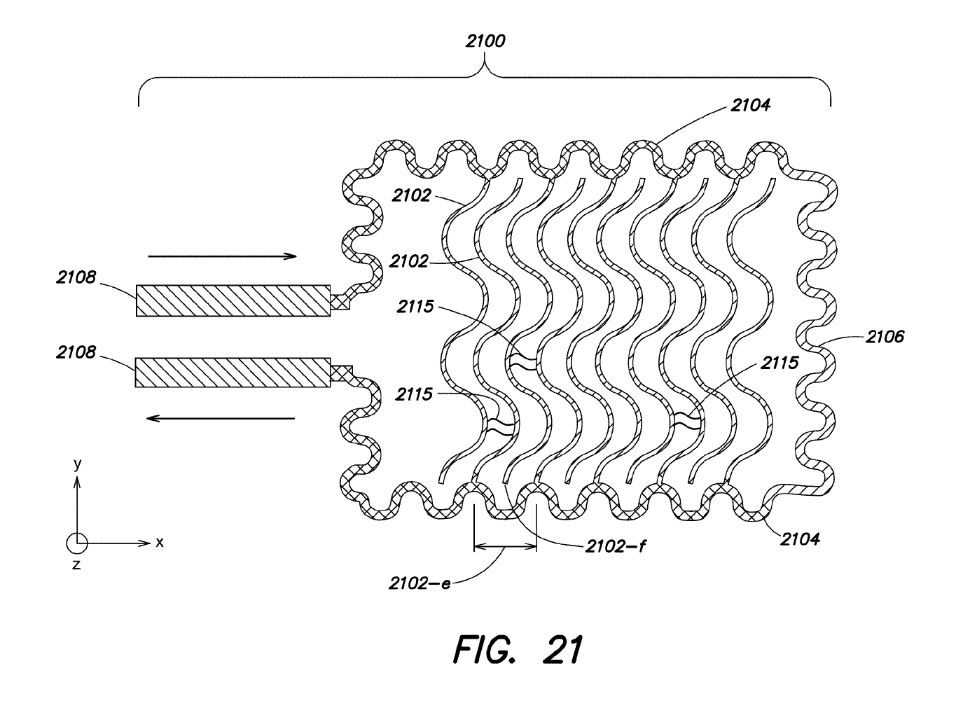

FIG. 20 shows the result of example measurements of a photodetector according to the principles herein based on a silicon substrate. The efficiency refers to the response to light falling exclusively on an exposed surface including that type. In an example, any area of the photodetector that is not intended to be exposed to electromagnetic radiation can be is covered with a high reflectivity material, such as but not limited to a metal.

The example photodetector of FIG. 20 has a substrate thickness of about 5 microns, a potential well thickness of about 3 microns, and a hole collector region and electron collector region of thickness about 0.6 microns. The QE decreases rapidly above about 500 nm, indicating that this example photodetector may perform better as a UV sensor than as an IR sensor. The minimal absorption above 450 nm means a 5 μm film can be close to transparent in appearance, with a red to yellow tinge to the human eye.

With knowledge of the difference between the hole collector region/electron collector region and the potential well region, an algorithm can be calibrated to the data to distinguish the separate components of the amount of UVA and amount UVB absorbed in the signal.

In another example, a filter can be deposited by repetitive deposition of dielectrics, to create a Fabry-Perot reflector. For instance, a sandwich of oxide/poly/oxide/poly/oxide/poly can be deposited to create a strong filter for a UVA wavelength, while another such sandwich structure of different layer thicknesses can be used for UVB wavelengths.

In an example implementation, a transistor based on this example silicon structure can be used to create a photo-pixel. The photo-current over a period of time can be integrated to build up a voltage. Such pixels can be used in digital photography, where the collection area is extremely small, and the photocurrents in a poorly lit room can be as low as 1 fA (10⁻¹⁵ amps). When higher optical currents are available, e.g. 1 nA (10⁻⁹ amps) where the collecting area is larger or there is more available illumination, the transistor can be used to build an on-island trans-impedance amplifier, which instantaneously provides a buffered voltage out proportional to the incoming photocurrent.

In an example implementation, the substrate 1802 can be formed based on a Group IV material, such as but not limited to silicon, silicon carbide, or germanium. The hole collector region 1808 can be formed from a highly acceptor doped region of the substrate, where the dopant is boron or gallium. The electron collector region 1806 can be formed from a highly donor doped region of the substrate, where the dopant is phosphorus or arsenic.

In another example implementation, the substrate 1802 can be formed from a Group III-V material, such as but not limited to gallium nitride, an indium gallium nitride, or an aluminum gallium nitride substrate. In this example, the dopant can be a Group IV element, such as silicon or germanium.

Hydration Sensors

As discussed above in connection with any of the example apparatus, systems or methods, the sensor component can be a hydration sensor. U.S. patent application Ser. No. 13/603,290, filed Sep. 4, 2012, and entitled “ELECTRONICS FOR DETECTION OF A CONDITION OF TISSUE,” which is hereby incorporated herein by reference in its entirety, describes hydration sensor that are applicable to any of the apparatus, systems and methods according to the principles described herein.

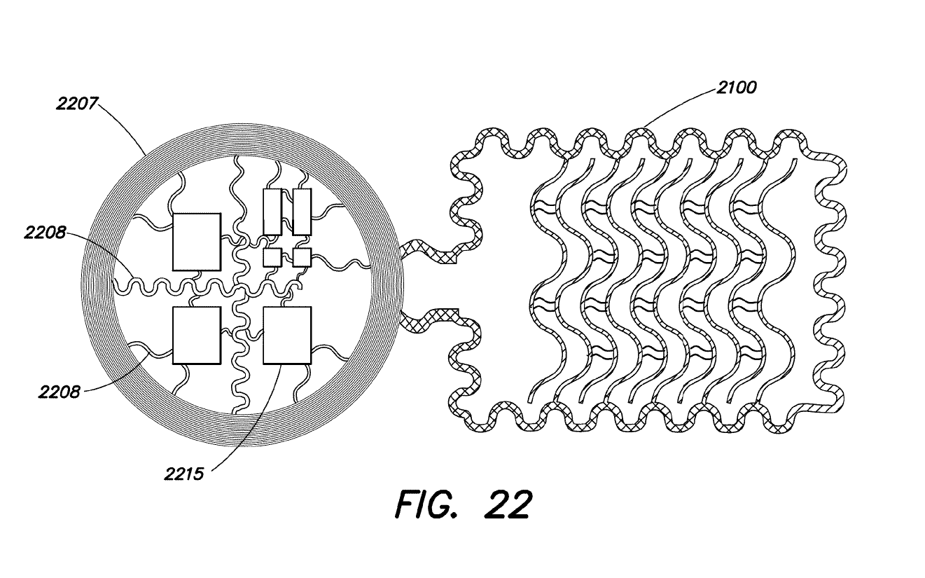

FIG. 21 shows a non-limiting example of a hydration sensor 2100 that interdigitated conductive structures 2102. The example apparatus 2100 can be disposed over the surface (such a but not limited to tissue) to perform the electrical measurements according to the principles described herein (which can be used to provide a measure of hydration level of the surface). A capacitance-based measurement can be performed by applying a potential across the interdigitated conductive structures. In the example of FIG. 21, the interdigitated conductive structures 2102 are disposed substantially parallel to each other. Each of the interdigitated conductive structures 2102 has a non-linear configuration. In the example of FIG. 21, the conductive structures 2102 have a serpentine configuration. In other examples, non-linear configuration of the conductive structures 2102 can be a, a zig-zag configuration, a rippled configuration, or any other non-linear configuration. The non-linear configuration of the conductive structures can facilitate greater sampling of the electrical properties of the tissue and higher signal to noise than linear electrodes. The non-linear configuration of the conductive structures also facilitates more consistent performance of the apparatus with deformation such as stretching. The example apparatus 2100 also includes two conductive brace structures 2104, each disposed substantially perpendicularly to the overall orientation of the interdigitated conductive structures 2102, and at least one spacer structure 2106 that is physically coupled at each of its ends to a portion of each of the at least two conductive brace structures. Each of the conductive brace structures 2104 is in electrical communication with alternating ones of the conductive structures 2102. For example, conductive structures 2102-e are in electrical communication with one of the conductive brace structure 2104 while the alternating, interposed conductive structure 2102-f is not in electrical communication with that conductive brace structure 2104. The spacer structure 2106 facilitates maintaining a substantially uniform separation between the brace structures 2104. The spacer structure 2106 can also facilitates maintaining a substantially uniform form factor during deformation of the apparatus. A measure of the electrical property of tissue using the example apparatus 2100 can be used to provide an indication of the condition of the tissue according to any of the principles described herein.

The conductive structures and the brace structures can include any applicable conductive material in the art, including a metal or metal alloy, a doped semiconductor, or a conductive oxide, or any combination thereof. Non-limiting examples of metals include Al or a transition metal (including Au, Ag, Cr, Cu, Fe, Ir, Mo, Nb, Pd, Pt, Rh, Ta, Ti, V, W or Zn), or any combination thereof. Non-limiting examples of doped semiconductors include any conductive form of Si, Ge, or a Group III-IV semiconductor (including GaAs, InP). In an example, the conductive structures and the brace structures can be formed from the same conductive material. In another example, the conductive structures and the brace structures can be formed from different conductive materials.

The conductive structures and/or the brace structures may be covered on at least one side by a polymer-based material, such as but not limited to a polyimide. In an example, the conductive structures and/or the brace structures may be encased in the polymer-based material. The polymer-based material can serve as an encapsulant layer.

Spacer structure also may be formed from a polymer-based material.

Apparatus 2100 or a system that includes apparatus 2100 may include a protective and/or backing layer made of a stretchable and/or flexible material. Non-limiting examples of materials that can be used for the protective and/or backing layer include any applicable polymer-based materials, such as but not limited to a polyimide or a transparent medical dressing, e.g., TEGADERM® (3M, St. Paul, Minn.). The protective and/or backing layer can include an adhesive portion that adheres to a portion of the substrate to assist in maintaining the conductive structures 2102 in contact with the substrate (including the tissue).

In a non-limiting example, the dimensions and morphology of the sensing component can be maintained using the spacer structure 2106. In an example, the spacer structure 2106 is formed from an insulating material or another material with lower conductivity than the conductive structures or the brace structures. The properties of the spacer structure 2106 of the apparatus 2100 can facilitate little or no current directly passing from one brace structure to the other brace structure by way of the spacer structure 2106. Rather, current passes from one set of the conductive structures 2102 to another set of the conductive structures 2102 by way of the underlying tissue.

In an example according to FIG. 21, the length of the ripples of the brace structure may be uniform or may vary from one side of the apparatus 2100 relative to the other.

In a non-limiting example, the non-linear configuration of the conductive structures facilitates increased flexibility of the apparatus. For example, the non-linear geometry can facilitate increased flexibility of the apparatus to stretching, torsion or other deformation of the underlying tissue, and the apparatus maintains substantial contact with the tissue in spite of the stretching, torsion or other deformation.

The apparatus 2100 includes cross-link structures 2115 that can be formed according to the principles herein. The cross-link structures 2115 can provide increased mechanical stability of the structure during fabrication (e.g., during a transfer process from a substrate and/or a printing and extraction process to another substrate), and in use, e.g., to stabilize the sensor against stretching, flexing, torsion or other deformation of the substrate it is disposed on. For example, the cross-link structures 2115 can aid in maintaining a form factor, including ratios of dimensions, during and/or after a stretching, elongation or relaxing of the apparatus. For example, the cross-link structures 2115 can be formed across any pair of the conductive structures 2102 of FIG. 21, at any position along their length. In the examples shown, the cross-links structures 2115 are formed in a serpentine (“S”) shape. In other examples, the cross-link structures 2115 can be formed as substantially straight crossbars, formed in a zig-zag pattern, formed as arcs, or ripples, or any other morphology that facilitates maintaining a mechanical stability and/or a form factor of the apparatus. In addition, the cross-link structures 2115 can be formed as at least two cross-link structures that are formed across neighboring electrodes. The cross-link structures 2115 can be formed from a polymer-based material or any other stretchable and/or flexible material. In addition, while the positioning of the example cross-link structures 2115 are shown to be roughly aligned in the x-direction of FIG. 21, cross-link structures 2115 also can be displaced relative to each other in the x-direction.

The cross-link structures 2115 can be formed of substantially the same encapsulant material that covers portions of the interdigitated conductive structures, and extend seamlessly from them. In this example, these cross-link structures 2115 can be formed during the same process step that disposes the encapsulant polymer-based material on portions of the interdigitated conductive structures. In another examples, the cross-link structures 515 can be formed of a different material from the encapsulant material that covers portions of the interdigitated conductive structures.

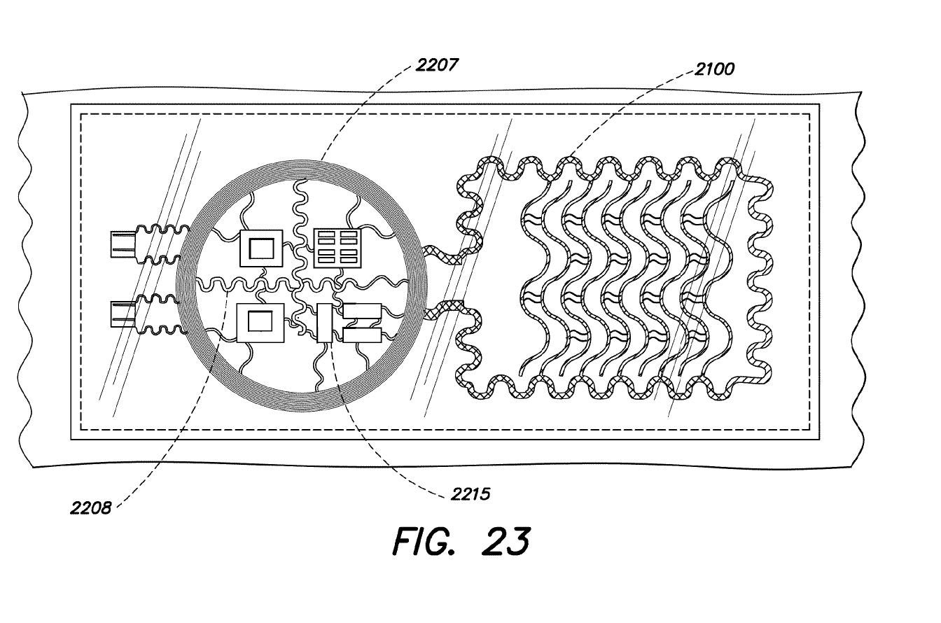

FIG. 22 shows the hydration sensor of FIG. 21 electrically coupled to an apparatus such as shown in FIG. 12A or 12B (and all related description). The apparatus includes a coil structure 2207, cross-link structures 2208, and at least one other component 2215. The at least one other component 2215 can be at least one of a battery, a transmitter, a transceiver, an amplifier, a processing unit, a charger regulator for a battery, a radio-frequency component, a memory, an analog sensing block, and a temperature sensor. Any description above in connection with the components or features of FIG. 11A, 11B, 12A, 12B, or 13 are also applicable to the equivalent features or components of FIG. 22. As shown in FIG. 22, cross-link structure physically couples a portion of the component 2215 to a portion of the coil structure 2207. The cross-link structures are formed from a flexible material that is non-conductive. In an example, the cross-link structures 2208 may link to portions of the coil structure 2207 that are closer to the center. In another example, the cross-link structures 2208 may link to the outer portions of the coil structure 2207. The measure of the amount of electromagnetic radiation incident on the sensor component 2204 provides an indication of the amount of exposure of the surface to the electromagnetic radiation.

In the example of FIG. 12A, the coil structure 1207 surrounds component 2215. In another example, the component 2215 may be positioned outside of the coil structure 2207. In another example, the component 2215 may be positioned outside the coil structure 2207.

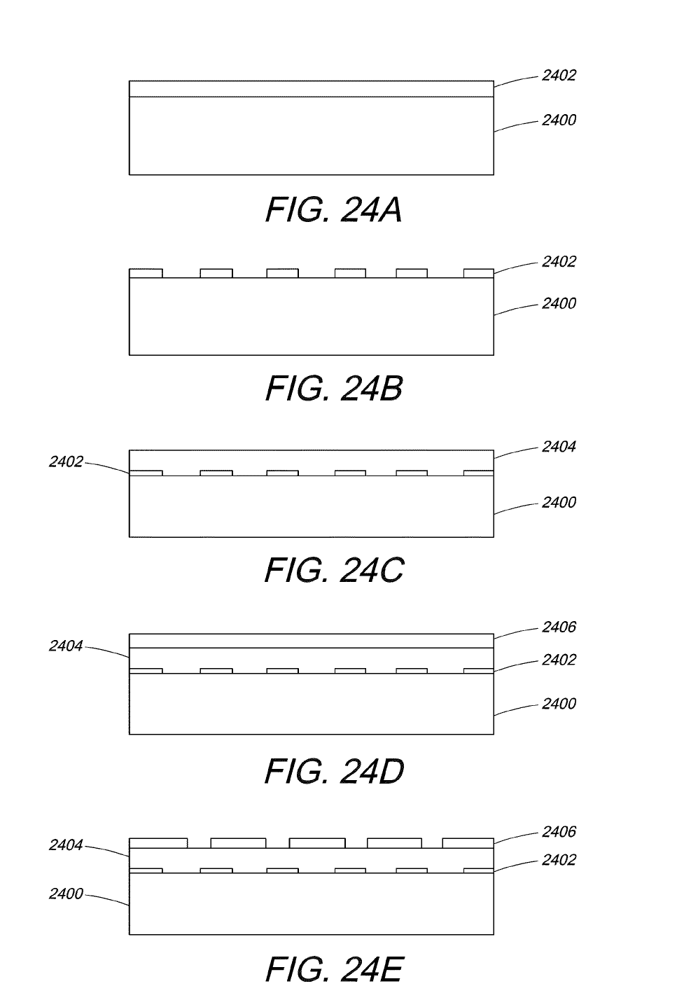

FIG. 23 shows an example implementation of a structure as described in connection with FIG. 22, which includes implementations of coil structure 2207, cross-link structures 2208, component 2215 and hydration sensor 2100.

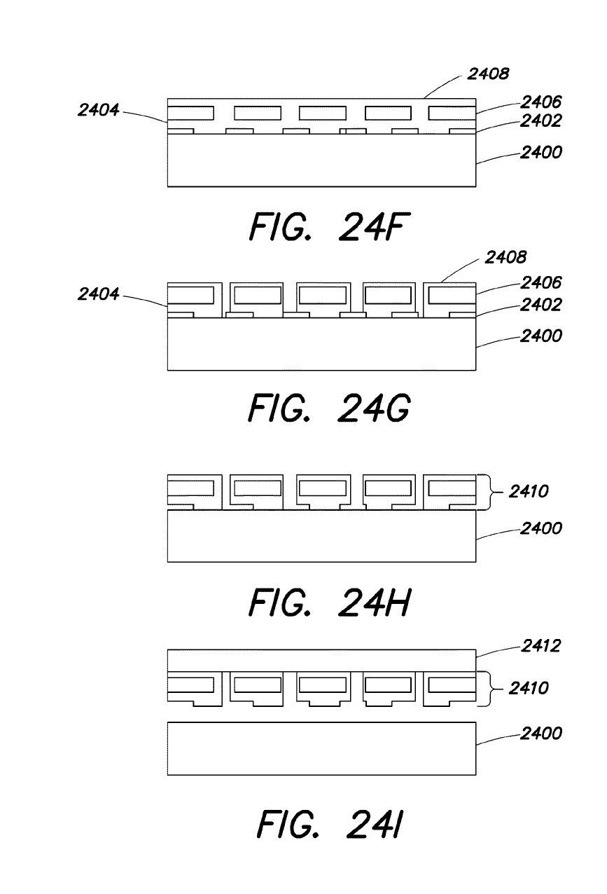

A non-limiting example process for fabricating any example apparatus or system described herein is illustrated in FIGS. 24A-24I. In FIG. 24A, a fabrication substrate 2400, such as but not limited to a group IV substrate (such as silicon) or a substrate for group III-V electronics, is coated with a with a sacrificial release layer 2402. In a non-limiting example, the sacrificial release layer 2402 is a polymer such as polymethylmethacrylate (PMMA). In FIG. 24B, the sacrificial release layer 2402 is patterned. In FIG. 24C, a first polymer layer 2404 is spin coated onto the sacrificial release layer 2402. In an example, the first polymer layer 2404 can be a polyimide. In FIG. 24D, a layer of conductive material 2406 is deposited over the first polymer layer 2404 to form the conductive structures. In FIG. 24E, where applicable to the conductive material 2406 used, a lithography process may be performed to pattern the conductive material 2406 into any of the configurations of conductive components described herein. In FIG. 24F, a second polymer layer 2408 is spin coated over the conductive components. In an example, the second polymer layer 2408 can be a polyimide. In FIG. 24G, the second polymer layer 2408 is patterned. In FIG. 24H, the sacrificial release layer material is selectively removed. For example, where the sacrificial release layer material is PMMA, acetone can be used for selective removal. At this stage, the apparatus 2410 is in substantially final form and attached to the fabrication substrate. In FIG. 24I, a transfer substrate 2412 is used to remove the apparatus 2410 from the fabrication substrate 2400.

### Example System and Communication

Also provided herein is an electromagnetic radiation (UV/sunlight/IR) exposure monitoring patch which operates by measuring either total visible sunlight or directly measuring UV light by means of one or more photodiodes. The output of the photodiode(s) can then be stored on the device into solid-state memory and/or transmitted off of the device through radio frequency (RF) communication.

The device may have multiple implementations based on the power source and data communication method. The implementations may have any combination of the following components:

1. Power storage, including but not limited to solid-state batteries, thin film batteries, or coin-cell batteries

2. Power generation, including but not limited to photovoltaic, kinetic, thermoelectric, radio frequency, or inductive coupling.

3. Communication, including but not limited to radio frequency, wires, or optical.

4. Data storage, including but not limited to solid-state memory.

These core attributes may be obtained “off-the shelf, then ground down (thinned) to microns or tens of microns thickness, or configured to maintain their ‘off the shelf’ physical dimensions prior to assembly with the “patch.” The final “patch” form factor may be implemented by assembling the components into a final package that is made to be thin and deformable (flexible, bendable, and/or stretchable).

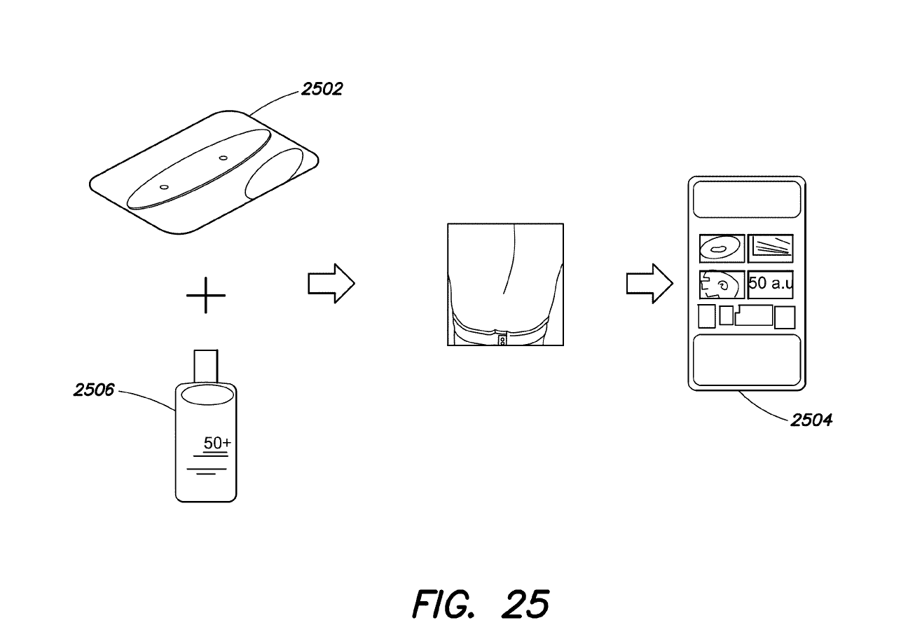

In an non-limiting example, an apparatus or system according to any of the principles described herein can be mounted to the surface as a part of a patch. The surface can be a part of a surface of paper, wood, leather, fabric (including artwork or other works on canvas), a plant or a tool. An example of a patch 2502 that can include at least one of any of the apparatus described herein is shown in FIG. 25. The patch 2502 may be applied to the surface, such as skin. A handheld device 2504 can be used to read the data in connection with the electrical measurement performed by the apparatus of the patch 2502. For example, the patch 2502 can include a transmitter or transceiver to transmit a signal to the handheld device 2504. The data in connection from the sensor component can be analyzed as described hereinabove by a processor of the handheld device 2502 to provide the indication of the exposure of the surface to electromagnetic radiation, the SPF factor of a product, or a condition of the surface according to the principles described herein.

As shown in FIG. 25, the patch may be used in connection with a substance 2506 that is applied to the surface. The substance 2506 may be configured to change the condition of the surface, including treating a disease of the surface. For example, the substance 2506 may be configured to be applied to the surface to provide protection against the UV. In this example, the apparatus of the patch would be configured to perform electrical measurements to provide an indication of UV and/or SPF sensing on the surface, to prevent sun damage and/or to recommend protective products. In another example, the substance 2506 may be configured to be applied to the surface to treat a disease or other malformation of the surface.

In an example, the patch 2502 may be a disposable adhesive patch that is configured for comfort and breathability.

In another example, the patch 2502 may be a more durable sensor patch that is configured for comfort and long-term wear. The sensor patch may include onboard sensors to measure the condition of interest of the surface, a memory to log the data in connection with the electrical communication, and a near-field communication device that allows a scan of the sensor patch with a handheld device to perform a status check and download. Non-limiting examples of the handheld device include a smartphone, tablet, slate, an e-reader or other handheld computing device. The sensor patch may include an energy storage device, such as a battery, to provide the voltage potential used for performing the measurements as described hereinabove.

In an example, the system may include the patch 2502 and a charging pad (not shown). The patch 2502 may be placed on the charging pad to charge the energy storage component of the patch 2502. The charging pad may be charged in an AC wall socket. The charging pad may be an inductive charging pad.

In an example implementation, the patch 2502 can include an apparatus for performing SPF monitoring based on the electrical information from a capacitance-based and/or an inductance-based measurement. The example apparatus according to this implementation can include an onboard UVA and/or UVB sensor. The condition of the surface that is reported is the sun protection effectiveness of a sunscreen product for protection of the surface. An example disposable patch according to this implementation can provide a surface that is engineered to simulate skin wetting properties to, accurately represent sunscreen distribution.

The example SPF monitoring system can use a durable sensor patch along with disposable adhesive patches. In an example method for use of the SPF monitoring system, the patch 2502 can be placed in a discreet high-exposure location on a person's body if extended sun exposure is expected. Over time, e.g., throughout the day, a NFC-enabled handheld device can be placed in proximity to the patch 2502 to check how much sun protection still remains. The handheld device can include an application (an App) to log and track “SPF state.” That is, the App on the handheld device can include machine-readable instructions such that a processor unit of the handheld device analyzes the electrical measurements from the apparatus of the patch 2502 and provides the indication of the status (SPF state) based on the analysis. The App can include machine-readable instructions to provide (i) product recommendations, (ii) suggestions to re-apply a product, or (iii) present an interface that facilitates the purchase of, or obtaining a sample of, recommended products. After use, such as at the end of the day, a consumer may dispose of the Adhesive patch, and retain the sensor patch reuse at a later time. The sensor patch can be re-charged using a charging pad as described herein.

In another example implementation, the patch 2502 can include an apparatus to perform as a UV dosimeter based on the electrical information from a capacitance-based and/or an inductance-based measurement. The example apparatus according to this implementation can include an onboard UVA and/or UVB sensor. The condition that is reported is the UV dosage exposure of an individual.

The example UV dosimeter system can use a durable sensor patch along with disposable adhesive patches. In an example method for use of the UV dosimeter system, the patch 2502 can be placed in a discreet high-exposure location on a person's body if extended sun exposure is expected. Over time, e.g., throughout the day, a NFC-enabled handheld device can be brought in proximity to the Adhesive patch to download logged data, gathered throughout use of the patch 2502. The App can be used to track “personal sun exposure state.” That is, the App on the handheld device can include machine-readable instructions such that a processor unit of the handheld device analyzes the electrical measurements from the apparatus of the patch 2502 and provides the indication of the status (personal sun exposure state) based on the analysis. The App can include machine-readable instructions to provide and can provide (i) product recommendations, (ii) suggestions to re-apply products, or (iii) present an interface that facilitates the purchase of, or obtaining a sample of, recommended products. After use, such as at the end of the day, the individual may dispose of the Adhesive patch, and retain the sensor patch for reuse at a later time. The sensor patch can be re-charged on charging pad, e.g., overnight.

In another example implementation, the patch 2502 can include an apparatus to perform as a hydration and/or firmness monitor based on the electrical information from a capacitance-based and/or an inductance-based measurement. The example apparatus according to this implementation can include an onboard hydration sensor. The condition that is reported is the hydration and/or firmness of a surface. Based on the indication, the patch 2502 can perform diagnosis and recommendation for personalized skin hydration and firmness product treatments.

The example hydration and/or firmness monitoring system can use a durable sensor patch along with disposable adhesive patches. In an example method for use of the hydration and/or firmness monitoring system, the individual may create a personal profile and affiliate a product choice with that profile on a handheld device. An App that can be used to generate the profile may be downloaded to the handheld device. After application of a product, e.g., at night, an individual may place one or more patches 2502 on an area of interest on the body. The individual may bring the NFC-enabled handheld device in proximity to the patch(es) 2502 to download data gathered intermittently during use of the patch(es) 2702. The App can include machine-readable instructions to track “personal hydration and firmness states.” In another example, the App can include machine-readable instructions to provide (i) product recommendations, (ii) suggestions to re-apply products, or (iii) present an interface that facilitates purchase of, or obtaining a sample of, recommended products. The individual may repeat the procedure with varying products and beauty routines and update the profile based on the results.

### CONCLUSION

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described examples of the invention can be implemented in any of numerous ways. For example, some examples may be implemented using hardware, software or a combination thereof. When any aspect of an example is implemented at least in part in software, the software code can be executed on any suitable processor or collection of processors, whether provided in a single device or computer or distributed among multiple devices/computers.

In this respect, various aspects of the invention, may be embodied at least in part as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various examples of the technology discussed above. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this example, one or more computer programs that when executed perform methods of the present technology need not reside on a single computer or processor, but may be distributed in a modular fashion amongst a number of different computers or processors to implement various aspects of the present technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various examples.

Also, the technology described herein may be embodied as a method, of which at least one example has been provided. The acts performed as part of the method may be ordered in any suitable way. Accordingly, examples may be constructed in which acts are performed in an order different than illustrated, which may include performing some acts simultaneously, even though shown as sequential acts in illustrative examples.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one example, to A only (optionally including elements other than B); in another example, to B only (optionally including elements other than A); in yet another example, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one example, to at least one, optionally including more than one, A, with no B present (and optionally including elements other than B); in another example, to at least one, optionally including more than one, B, with no A present (and optionally including elements other than A); in yet another example, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All examples that come within the spirit and scope of the following claims and equivalents thereto are claimed.

What is claimed is:

1. An apparatus for monitoring exposure of a surface to electromagnetic radiation, the apparatus comprising:
a flexible substrate;
at least one sensor component disposed on the flexible substrate, wherein the at least one sensor component measures an amount of electromagnetic radiation incident on the at least one sensor component, the electromagnetic radiation having frequencies in the visible or ultraviolet regions of the electromagnetic spectrum;
at least one processing unit in communication with the at least one sensor component; and
at least one cross-link structure physically coupled to a portion of the at least one processing unit and/or to a portion of the at least one sensor component, the at least one cross-link structure being formed from a dielectric material,
wherein the measure of the amount of electromagnetic radiation incident on the at least one sensor component provides an indication of an amount of exposure of the surface to the electromagnetic radiation.

2. The apparatus of claim 1, wherein the at least one cross-link structure physically couples a portion of the at least one processing unit to a portion of the at least one sensor component.

3. The apparatus of claim 1, further comprising a memory in communication with the at least one sensor component, wherein the memory stores data indicative of measurements of the amount of electromagnetic radiation incident on the at least one sensor component.

4. The apparatus of claim 1, further comprising a memory in communication with the at least one sensor component, wherein the memory stores machine readable instructions which, when executed, cause the at least one processing unit to analyze the measure of the amount of electromagnetic radiation incident on the at least one sensor component to provide the indication of the amount of exposure of the surface to the electromagnetic radiation.

5. The apparatus of claim 1, further comprising at least one coil structure formed from a conductive material, and a radio-frequency component in communication with the at least one coil structure and/or the at least one processing unit, wherein the radio-frequency component transmits the measure of the amount of electromagnetic radiation incident on the at least one sensor component and/or the indication of an amount of exposure of the surface to the electromagnetic radiation using the at least one coil structure.

6. The apparatus of claim 1, the radio-frequency component is a BLUETOOTH® component.

7. The apparatus of claim 1, further comprising:
at least one brace structure formed from a dielectric material;
wherein the at least one cross-link structure physically couples a portion of the at least one processing unit and/or a portion of the at least one sensor component to the at least one brace structure.

8. The apparatus of claim 7, wherein the at least one brace structure and the at least one cross-link structure are formed from the same material or formed from different materials.

9. The apparatus of claim 7, wherein the at least one brace structure surrounds the at least one processing unit and/or the at least one sensor component.

10. The apparatus of claim 7, wherein the flexible substrate and the at least one cross-link structure are formed from the same material or formed from different materials.

11. The apparatus of claim 7, wherein the flexible substrate and the at least one cross-link structure are formed from a same polymer.

12. The apparatus of claim 7, wherein the flexible substrate has a Young's modulus of less than about 10 GPa.

13. The apparatus of claim 1, further comprising an encapsulation layer disposed over at least a portion of the at least one sensor component and/or at least a portion of the at least one processing unit.

14. The apparatus of claim 13, wherein the at least one sensor component and the at least one processing unit are positioned at or near a midpoint of a depth of the apparatus.

15. The apparatus of claim 13, wherein the encapsulation layer has a Young's modulus less than about 100 MPa.

16. The apparatus of claim 13, wherein portions of the encapsulation layer comprise an adhesive, and wherein the adhesive attaches the portions of the encapsulation layer to the surface.

17. The apparatus of claim 13, wherein the encapsulation layer is formed from a polymer.

18. The apparatus of claim 1, wherein the at least one sensor component is a photodetector comprising a p-n junction.

19. The apparatus of claim 1, further comprising at least one filter disposed above the at least one sensor component, and wherein a measure of the electromagnetic radiation using the at least one filter and the at least one sensor component provides a measure of the amount of ultraviolet-A electromagnetic radiation and/or ultraviolet-B electromagnetic radiation incident on the surface.

20. The apparatus of claim 1, wherein the at least one sensor component is at least partially embedded in the flexible substrate.

21. The apparatus of claim 1, wherein the at least one sensor component comprises two sensor component, and wherein one of the two sensor components is stacked above the other of the two sensor components to provide a stacked sensor component.

22. The apparatus of claim 21, wherein a comparison of a measure of the electromagnetic radiation using the stacked sensor component to a measure of the electromagnetic radiation using another of the at least one sensor components provides a measure of the amount of ultraviolet A electromagnetic radiation and/or ultraviolet B electromagnetic radiation incident on the surface.

23. The apparatus of claim 1, wherein the at least one sensor component comprises a photodetector.

24. The apparatus of claim 23, wherein the at least one sensor component is at least one of a silicon-based photodetector, a silicon carbide-based photodetector, a germanium-based photodetector, a gallium nitride-based photodetector, an indium gallium nitride-based photodetector and an aluminum gallium nitride-based photodetector.

25. The apparatus of claim 1, wherein the surface is a portion of a tissue, a fabric, a plant, an artwork, paper, wood, or a tool or piece of equipment.

26. The apparatus of claim 25, wherein the surface is a portion of a tissue, and wherein the measure of the amount of exposure of the surface of the tissue to the electromagnetic radiation provides a measure of a level of SPF protection of the tissue.

27. The apparatus of claim 26, wherein the at least one sensor component comprises at least two sensor components, wherein an ultraviolet filter is disposed above at least one of the at least two sensor components, and wherein a comparison of a measure of the electromagnetic radiation using the sensor component including the ultraviolet filter to a measure of the electromagnetic radiation using another of the at least one sensor components having no ultraviolet filter provides the measure of a level of SPF protection of the tissue.

28. The apparatus of claim 1, further comprising at least one amplifier in electrical communication with the at least one sensor component.

29. A system for monitoring exposure of a surface to electromagnetic radiation, the system comprising:
at least one apparatus of claim 1; and
a reader device,
wherein the reader device receives from the at least one apparatus the data indicative of the measure of the amount of electromagnetic radiation incident on the at least one sensor component and/or the indication of an amount of exposure of the surface to the electromagnetic radiation.

30. The system of claim 29, wherein the reader device comprises a coupling member, and wherein the reader device receives the data indicative of the measure of the amount of electromagnetic radiation incident on the at least one sensor component and/or the indication of an amount of exposure of the surface to the electromagnetic radiation when the coupling member is electrically coupled to a portion of the at least one apparatus.

31. The system of claim 29, wherein the surface is a portion of a tissue, a fabric, a plant, an artwork, paper, wood, or a tool or piece of equipment.

32. The system of claim 29, wherein the reader device is a near-field communication (NFC)-enabled handheld device.

33. An apparatus for monitoring exposure of a surface to electromagnetic radiation, the apparatus comprising:
at least one sensor component, wherein the at least one sensor component measures an amount of electromagnetic radiation incident on the at least one sensor component, the electromagnetic radiation having frequencies in the visible or ultraviolet regions of the electromagnetic spectrum;
at least one coil structure formed from a conductive material; and
at least one cross-link structure physically coupling a portion of the at least one coil structure to a portion of the at least one sensor component, the at least one cross-link structure being formed from a flexible material,
wherein the measure of the amount of electromagnetic radiation incident on the at least one sensor component provides an indication of an amount of exposure of the surface to the electromagnetic radiation.

34. The apparatus of claim 33, wherein the at least one sensor component is surrounded by the at least one coil structure.

35. The apparatus of claim 33, wherein the at least one sensor component is positioned outside the at least one coil structure.

36. The apparatus of claim 33, wherein the surface is a portion of a tissue, a fabric, a plant, an artwork, paper, wood, or a tool or piece of equipment.

37. The apparatus of claim 33, wherein the at least one sensor component is surrounded by the at least one coil structure.

38. The apparatus of claim 33, wherein the measure of the amount of exposure of the tissue to the electromagnetic radiation provides a measure of a level of SPF protection of the surface.

39. The apparatus of claim 33, further comprising at least one processing unit in communication with the at least one sensor component.

40. The apparatus of claim 39, wherein the at least one processing unit analyzes the measure of the amount of electromagnetic radiation incident on the at least one sensor component to provide the indication of the amount of exposure of the surface to the electromagnetic radiation.

41. The apparatus of claim 39, further comprising a radio-frequency component in communication with the at least one coil structure and the at least one processing unit, wherein the radio-frequency component transmits the measure of the amount of electromagnetic radiation incident on the at least one sensor component and/or the indication of an amount of exposure of the surface to the electromagnetic radiation using the at least one coil structure.

42. The apparatus of claim 33, wherein the at least one coil structure comprises at least one corrugated portion.

43. The apparatus of claim 42, wherein the at least one corrugated portion comprises a zig-zag structure, a serpentine structure, a grooved structure, or a rippled structure.

44. The apparatus of claim 33, wherein the at least one coil structure is polygonal-shaped, circular-shaped, square-shaped or rectangular-shaped.

45. The apparatus of claim 33, further comprising a flexible substrate, wherein the at least one sensor component and the at least one coil structure are disposed on the flexible substrate.

46. The apparatus of claim 45, wherein the flexible substrate is a polymer.

47. The apparatus of claim 46, wherein the at least one cross-link structure is formed from a polymer.

48. The apparatus of claim 46, wherein the flexible substrate and the at least one cross-link structure are formed from the same material or from different materials.

49. The apparatus of claim 46, wherein the flexible substrate and the at least one cross-link structure are formed from a same polymer.

50. The apparatus of claim 46, wherein the flexible substrate has a Young's modulus of less than about 10 GPa.

51. The apparatus of claim 33, wherein the at least one sensor component comprises a photodetector.

52. The apparatus of claim 51, wherein the at least one sensor component is at least one of a silicon-based photodetector, a silicon carbide-based photodetector, a germanium-based photodetector, a gallium nitride-based photodetector, an indium gallium nitride-based photodetector and an aluminum gallium nitride-based photodetector.

53. The apparatus of claim 51, further comprising a filter coupled to the at least one sensor component, wherein the filter is disposed at a region of the at least one sensor component where the electromagnetic radiation is incident.

54. The apparatus of claim 51, wherein a measure of a change in current of the photodetector provides the measure of the amount of electromagnetic radiation incident on the at least one sensor component.

55. The apparatus of claim 33, wherein the at least one sensor component measures the amount of ultraviolet (UV) electromagnetic radiation incident on the at least one sensor component.

56. The apparatus of claim 33, wherein the at least one sensor component measures the amount of UVA or UVB electromagnetic radiation incident on the at least one sensor component.

57. The apparatus of claim 33, further comprising an encapsulation layer disposed over at least a portion of the at least one sensor component and the at least one coil structure.

58. The apparatus of claim 57, wherein the encapsulation layer has a Young's modulus less than about 100 MPa.

59. The apparatus of claim 57, wherein the at least one sensor component is positioned at or near a midpoint of a depth of the apparatus.

60. The apparatus of claim 57, wherein portions of the encapsulation layer comprise an adhesive, and wherein the adhesive attaches the portions of the encapsulation layer to the surface.

61. The apparatus of claim 57, wherein the encapsulation layer is formed from a polymer.

62. The apparatus of claim 57, wherein the polymer is a polyimide, and wherein the at least one sensor component measures the amount of visible electromagnetic radiation incident on the apparatus.

63. The apparatus of claim 57, wherein the encapsulation layer is formed from an elastomer.

64. The apparatus of claim 57, wherein the encapsulation layer and the at least one cross-ink structures are formed from the same material.

65. A system for monitoring exposure of a surface to electromagnetic radiation, comprising:
at least one apparatus of claim 33; and
at least one other component,
wherein the at least one other component is at least one of a battery, a transmitter, a transceiver, an amplifier, a processing unit, a charger regulator for a battery, a radio-frequency component, a memory, an analog sensing block, and a temperature sensor.

66. A method for monitoring exposure of a surface to electromagnetic radiation, the method comprising:
receiving data indicative of the amount of electromagnetic radiation incident on the at least one sensor component, wherein the data is obtained using at least one apparatus of claim 33; and
analyzing the data using at least one processor unit, wherein the analysis provides indication of an amount of exposure of the surface to the electromagnetic radiation.

67. The method of claim 66, wherein the analyzing the data comprises comparing the data to a calibration standard, and wherein the comparing provides the indication of the amount of exposure of the surface to the electromagnetic radiation.

68. The method of claim 66, wherein the calibration standard comprises a correlation between values of the data and the indication of the amount of exposure of the surface to the electromagnetic radiation.

69. An electromagnetic radiation sensor, comprising:
a substrate having a surface that is exposed to electromagnetic radiation in the visible and ultraviolet regions of the electromagnetic spectrum;
an electron collector region disposed in the substrate;
a hole collector region disposed in the substrate; and
a potential well region disposed in the substrate and surrounding at least a portion of the electron collector region and at least a portion of the hole collector region.

70. The sensor of claim 69, wherein the electron collector region comprises a highly donor doped semiconductor material.

71. The sensor of claim 69, wherein the hole collector region comprises a highly acceptor doped semiconductor material.

72. The sensor of claim 69, wherein the potential well region comprises a donor doped semiconductor material and the substrate is a p-type semiconductor material, or wherein the potential well region comprises an acceptor doped semiconductor material and the substrate is a n-type semiconductor material.

73. The sensor of claim 72, wherein the potential well region comprises a donor doped semiconductor material and the substrate is a p-type semiconductor material, and wherein the potential well region comprises a lower concentration of a dopant than the electron collector region.

74. The sensor of claim 69, wherein the substrate comprises silicon, silicon carbide, germanium, gallium nitride, indium gallium nitride, or aluminum gallium nitride.

75. The sensor of claim 74, wherein the substrate comprises silicon, silicon carbide, or germanium, wherein the hole collector region is formed from a highly acceptor doped region of the substrate, and the hole collector region comprises a boron dopant or a gallium dopant.

76. The sensor of claim 74, wherein the substrate comprises silicon, silicon carbide, or germanium, wherein the electron collector region is formed from a highly donor doped region of the substrate, and wherein the electron collector region comprises a phosphorus dopant or an arsenic dopant.

77. The sensor of claim 75, wherein the substrate comprises silicon, silicon carbide, or germanium, wherein the potential well region is formed from a donor doped region of the substrate, wherein the potential well region has a lower concentration of dopant than the electron collector region, and wherein the potential well region comprises a phosphorus dopant or an arsenic dopant.

78. The sensor of claim 75, wherein the substrate comprises silicon, silicon carbide, or germanium, wherein the potential well region is formed from an acceptor doped region of the substrate, wherein the potential well region has a lower concentration of dopant than the hole collector region, and wherein the potential well region comprises a boron dopant or a gallium dopant.

79. The sensor of claim 69, wherein the electron collector region is disposed proximate to the surface of the substrate or embedded in the substrate.

80. The sensor of claim 69, wherein the hole collector region is disposed proximate to the surface of the substrate or embedded in the substrate.

81. The sensor of claim 69, wherein the substrate has a thickness of less than 1 micron, about 1 micron, about 2 micron, about 3 microns, about 5 microns, about 10 microns, or greater than about 10 microns.

82. The sensor of claim 69, wherein the potential well region has a thickness greater than the thickness of the electron collector region or the hole collector region.

83. The sensor of claim 82, wherein the electron collector region has a thickness of less than 1 micron, about 1 micron, about 2 microns, about 3 microns, or greater than about 3 microns.

84. The sensor of claim 82, wherein the hole collector region has a thickness of less than 1 micron, about 1 micron, about 2 microns, about 3 microns, or greater than about 3 microns.

85. The sensor of claim 69, wherein the potential well region has a thickness of less than 1 micron, about 1 micron, about 2 microns, about 3 microns, about 4 microns, or greater than about 4 microns.

86. The sensor of claim 69, wherein a portion of the potential well is disposed between the electron collector region and the hole collector region.

87. A system comprising:
at least one coil structure formed from a conductive material;
at least one other component, wherein the at least one other component is at least one of a battery, a transmitter, a transceiver, an amplifier, a processing unit, a charger regulator for a battery, a radio-frequency component, a memory, an analog sensing block, and a temperature sensor; and
at least one cross-link structure physically coupling a portion of the at least one coil structure to a portion of the at least one other component, the at least one cross-link structure being formed from a flexible material.

88. The system of claim 87, further comprising at least one sensor component.

89. The system of claim 88, wherein the at least one sensor component measures an amount of electromagnetic radiation incident on the at least one sensor component, the electromagnetic radiation having frequencies in the visible or ultraviolet regions of the electromagnetic spectrum.

90. The system of claim 89, wherein the system is disposed on a surface, and wherein the measure of the amount of electromagnetic radiation incident on the at least one sensor component provides an indication of an amount of exposure of the surface to the electromagnetic radiation.

91. The system of claim 88, wherein the at least one sensor component is positioned external to the at least one coil structure, and wherein the at least one sensor component is electrically coupled to the at least one coil structure or to the at least one other component.

92. The system of claim 88, wherein at least one other component or the at least one sensor component is surrounded by the at least one coil structure.

93. The system of claim 88, wherein the system is disposed on a tissue, and wherein the at least one sensor component measures a hydration level of the tissue.

94. The system of claim 88, wherein the at least one other component is a radio-frequency component and a processing unit, wherein the radio-frequency component is in communication with the at least one coil structure and the at least one processing unit, and wherein the radio-frequency component transmits data indicative of a measurement performed by the at least one sensor component.

95. The system of claim 88, wherein the at least one sensor component comprises a photodetector.

96. The system of claim 95, wherein the at least one sensor component is at least one of a silicon-based photodetector, a silicon carbide-based photodetector, a germanium-based photodetector, a gallium nitride-based photodetector, an indium gallium nitride-based photodetector and an aluminum gallium nitride-based photodetector.

97. The system of claim 95, further comprising a filter coupled to the at least one sensor component, wherein the filter is disposed at a region of the at least one sensor component where the electromagnetic radiation is incident.

98. The system of claim 95, wherein a measure of a change in current of the photodetector provides the measure of the amount of electromagnetic radiation incident on the at least one sensor component.

99. The system of claim 87, wherein the system is disposed on a surface, and wherein the surface is a portion of a tissue, a fabric, a plant, an artwork, paper, wood, or a tool or piece of equipment.

100. The system of claim 87, wherein the at least one coil structure comprises at least one corrugated portion.

101. The system of claim 100, wherein the at least one corrugated portion comprises a zig-zag structure, a serpentine structure, a grooved structure, or a rippled structure.

102. The system of claim 87, wherein the at least one coil structure is polygonal-shaped, circular-shaped, square-shaped or rectangular-shaped.

103. The system of claim 87, further comprising a flexible substrate, wherein the at least one sensor component and the at least one coil structure are disposed on the flexible substrate.

104. The system of claim 103, wherein the flexible substrate is a polymer.

105. The system of claim 104, wherein the at least one cross-link structure is formed from a polymer.

106. The system of claim 104, wherein the flexible substrate and the at least one cross-link structure are formed from the same material or different materials.

107. The system of claim 104, wherein the flexible substrate and the at least one cross-link structure are formed from a same polymer.

108. The system of claim 87, further comprising an encapsulation layer disposed over at least a portion of the at least one coil structure and the at least one other component.

109. The system of claim 108, wherein the at least one sensor component is positioned at or near a midpoint of a depth of the system.

110. The system of claim 108, wherein the system is disposed on a surface, and wherein portions of the encapsulation layer comprise an adhesive, and wherein the adhesive attaches the portions of the encapsulation layer to the surface.

111. The system of claim 108, wherein the encapsulation layer is formed from a polymer.
